(19) United States
(12) Patent Application Publication
(10) Pub. No.: US 2007/0122396 A1
(43) Pub. Date: May 31, 2007

(54) Morphogenic proteins and stimulatory factors in gene therapy

(76) Inventors: John C. Lee, San Antonio, TX (US); Lee-Chuan C. Yeh, San Antonio, TX (US)

Correspondence Address: FISH & NEAVE IP GROUP;ROPES & GRAY LLP, 1211 AVENUE OF THE AMERICAS, NEW YORK, NY, 10036-8704, US

(21) Appl. No.: 11/595,354

(22) Filed: Nov. 8, 2006

Related U.S. Application Data
(63) Continuation of application No. PCT/US05/16426, filed on May 11, 2005.
(60) Provisional application No. 60/570,388, filed on May 11, 2004.

(51) Int. Cl. A61K 48/00; A61K 38/22; A61K 38/28; A61K 38/20; A61K 38/18

(52) U.S. Cl. 424/93.21; 514/12; 514/44; 514/3; 424/85.2

(57) ABSTRACT

Gene therapy methods for tissue formation, repair and regeneration using nucleic acids encoding morphogenic proteins and morphogenic protein stimulatory factors (MPSFs) are provided.

This application is a continuation of International application No. PCT/US2005/016426, filed May 11, 2005, the entire disclosure of which is incorporated by reference herein.

### TECHNICAL FIELD OF THE INVENTION

The present invention relates to gene therapy methods for tissue formation, repair and regeneration using nucleic acids encoding morphogenic proteins and morphogenic protein stimulatory factors.

### BACKGROUND OF THE INVENTION

Osteogenic and bone morphogenetic proteins represent a family of structurally and functionally related morphogenic proteins belonging to the Transforming Growth Factor-Beta (TGF-β) superfamily. The TGF-β superfamily, in turn, represents a large number of evolutionarily conserved proteins with diverse activities involved in growth, differentiation and tissue morphogenesis and repair. BMPs and osteogenic proteins, as members of the TGF-β superfamily, are expressed as secretory polypeptide precursors which share a highly conserved bioactive cysteine domain- located near their C-termini.

Many morphogenic proteins belonging to the BMP family have now been described. Some have been isolated using purification techniques coupled with bioassays such as the one described above. Others have been identified and cloned by virtue of DNA sequence homologies within conserved regions that are common to the BMP family. These homologs are referred to as consecutively-numbered BMPs whether or not they have demonstrable osteogenic activity. Using an alternative approach, synthetic OPs having osteogenic activity have been designed using amino acid consensus sequences derived from sequence comparisons between naturally-derived OPs and BMPs (see below; Oppermann et al., U.S. Pat. No. 5,324,819).

While several of the earliest members of the BMP family were osteogenic proteins identified by virtue of their ability to induce new cartilage and bone, the search for BMP-related genes and gene products in a variety of species has revealed new morphogenic proteins, some of which have different or additional tissue-inductive capabilities. For example, BMP-12 and BMP-13 (identified by DNA sequence homology) reportedly induce tendon/ligament-like tissue formation in vivo (WO 95/16035). Several BMPs can induce neuronal cell proliferation and promote axon regeneration (WO 95/05846). And, some BMPs that were originally isolated on the basis of their osteogenic activity also have neural inductive properties (Liem et al., Cell, 82, pp. 969-79 (1995)). It, thus, appears that osteogenic proteins and other BMPs may have a variety of potential tissue inductive capabilities whose final expression may depend on a complex set of developmental and environmental cues. These osteogenic, BMP and BMP-related proteins are referred to herein collectively as morphogenic proteins.

The activities described above, and other as yet undiscovered tissue inductive properties of the morphogenic proteins belonging to the BMP family are expected to be useful for promoting tissue regeneration in patients with traumas caused, for example, by injuries or degenerative disorders. Given the large number of potential therapeutic uses for morphogenic proteins in treating a variety of different tissues and tissue-types, there is a need for improved methods for inducing tissue repair and regeneration using these proteins.

### SUMMARY OF THE INVENTION

The present invention is based on the determination that progenitor cells may be genetically-engineered to produce proteins. In one embodiment, the invention provides methods for generating genetically-engineered progenitor cells. In one embodiment, the invention provides a method for inducing a progenitor cell to proliferate or differentiate comprising the steps of contacting a progenitor cell with a nucleic acid encoding a morphogenic protein and a morphogenic protein stimulatory factor (MPSF). In another embodiment, the invention provides a method for inducing a progenitor cell to proliferate or differentiate comprising the steps of: a) providing a vector comprising a nucleic acid encoding a morphogenic protein operably linked to an expression control sequence and a vector comprising a nucleic acid encoding a MPSF operably linked to an expression control sequence, and b) contacting said progenitor cell with said vectors.

In some embodiments, the invention provides gene therapy methods for inducing tissue formation, repairing a tissue defect or regenerating tissue at a target locus. In some embodiments, the invention provides a method for inducing tissue formation, repairing a tissue defect or regenerating tissue, at a target locus in a mammal, comprising the step of administering to the target locus a nucleic acid encoding a morphogenic protein and a nucleic acid encoding a MPSF. In other embodiments, the invention provides a method for inducing tissue formation, repairing a tissue defect or regenerating tissue, at a target locus in a mammal, comprising the steps of: a) providing a vector comprising a nucleic acid encoding a morphogenic protein operably linked to an expression control sequence and a vector comprising a nucleic acid encoding a MPSF operably linked to an expression control sequence, and b) administering to the target locus said vector. In yet other embodiments, the invention provides a method for inducing tissue formation, repairing a tissue defect or regenerating tissue, at a target locus in a mammal, comprising the steps of: a) providing a cultured host cell expressing a recombinant morphogenic protein and a recombinant MPSF, and b) administering to the target locus the host cell expressing the recombinant morphogenic protein and the recombinant MPSF.

In some embodiments, the invention provides a method of inducing tissue formation, repairing a tissue defect or regenerating tissue, by in vivo gene therapy, comprising the step of administering to target locus in a patient, a viral vector comprising a nucleotide sequence that encodes a morphogenic protein and a viral vector comprising a nucleotide sequence that encodes a MPSF so that the morphogenic protein and MPSF are expressed from the nucleotide sequence in the mammal in an amount sufficient to induce progenitor cells to proliferate or differentiate. In some embodiments, the viral vector includes but is not limited to an adenoviral vector, a lentiviral vector, a baculoviral vector, an Epstein Barr viral vector, a papovaviral vector, a vaccinia viral vector, and a herpes simplex viral vector.

In some embodiments of the invention, the nucleic acid encoding the morphogenic protein and the nucleic acid encoding the MPSF are in the same vector. In other embodiments, the nucleic acid encoding the morphogenic protein and the nucleic acid encoding the MPSF are in separate vectors.

In some embodiments of the invention, the morphogenic protein and MPSF are expressed in separate cells. In other embodiments of the invention, the morphogenic protein and MPSF are expressed in the same cell.

The progenitor cell that is induced to proliferate and/or differentiate by the morphogenic protein and MPSF of this invention is preferably a mammalian cell. Preferred progenitor cells include but are not limited to mammalian chondroblasts, osteoblasts, ligament progenitor cells, tendon progenitor cells and neuroblasts, all earlier developmental precursors thereof, and all cells that develop therefrom (e.g., chondroblasts, pre-chondroblasts and chondrocytes). However, morphogenic proteins are highly conserved throughout evolution, and non-mammalian progenitor cells are also likely to be stimulated by same- or cross-species morphogenic proteins and MPSF combinations.

In some embodiments, the target locus includes but is not limited to bone, cartilage, tendon, ligament and neural tissue.

In some embodiments, the invention provides a method for improving the tissue inductive activity in a mammal of a morphogenic protein capable of inducing tissue formation at a target locus by coadministering an effective amount of MPSF, the method comprising administering to the target locus a nucleic acid encoding the morphogenic protein and a nucleic acid encoding the MPSF.

The invention also provides a method of improving the tissue inductive activity in a mammal of a morphogenic protein capable of inducing tissue formation at a target locus by coadministering an effective amount of a MPSF, the method comprising administering to the target locus a vector comprising a nucleic acid encoding the morphogenic protein operably linked to an expression control sequence and a vector comprising a nucleic acid encoding the MPSF operably linked to an expression control sequence.

The invention also provides a method for improving the tissue inductive activity in a mammal of a morphogenic protein capable of inducing tissue formation at a target locus by coadministering an effective amount of a MPSF, the method comprising administering to the target locus a cell comprising a vector comprising a nucleic acid encoding the morphogenic protein operably linked to an expression control sequence and a cell comprising a vector comprising a nucleic acid encoding the MPSF operably linked to an expression control sequence. In some embodiments, the MPSF synergistically enhances the tissue inductive activity of the morphogenic protein.

In some embodiments, the nucleic acids encoding the morphogenic protein and the MPSF are in the same vector. In some embodiments, the nucleic acids encoding the morphogenic protein and the MPSF are in separate vectors. In some embodiments, the vectors comprising the nucleic acids encoding the morphogenic protein and the MPSF are in the same cell. In some embodiments, the vectors comprising the nucleic acids encoding the morphogenic protein and the MPSF are in separate cells.

In some embodiments, the morphogenic protein includes but is not limited to OP-1 (BMP-7), OP-2, OP-3, COP-1, COP-3, COP-4, COP-5, COP-7, COP-16, BMP-2, BMP-3, BMP-3b, BMP-4, BMP-5, BMP-6, BMP-9, BMP-10, BMP-11, CDMP-3, BMP-12, CDMP-2, BMP-13, CDMP-1, BMP-14, BMP-15, BMP-16, BMP-17, BMP-18, GDF-1, GDF-2, GDF-3, GDF-5, GDF-6, GDF-7, GDF-8, GDF-9, GDF-10, GDF-11, GDF-12, MP121, dorsalin-1, DPP, Vg-1, Vgr-1, 60A protein, NODAL, UNIVIN, SCREW, ADMP, NEURAL, or fragments thereof. In some embodiments, the morphogenic protein comprises a dimeric protein having an amino acid sequence having at least 70% homology within the C-terminal 102-106 amino acids of human OP-1. In some embodiments, the morphogenic protein is OP-1 or a fragment thereof.

A MPSF according to this invention is a factor that is capable of stimulating the ability of a morphogenic protein to induce tissue formation from a progenitor cell. In some embodiments, the MPSFs of this invention include hormones, cytokines and growth factors. Preferred MPSFs include but are not limited to insulin-like growth factor I (IGF-I), insulin-like growth factor II (IGF-II), fibroblast growth factor (FGF), growth hormone, insulin, parathyroid hormone (PTH), IL-6 or IL-6 together with soluble IL-6R (IL-6/IL-6R). A more preferred MPSF is IGF-I. Another more preferred MPSF is IL-6. Another more preferred MPSF is IL-6 together with soluble IL-6R.

### BRIEF DESCRIPTION OF THE DRAWINGS

FIG. 1 depicts (A) Western blot analysis of OP-1 expression in FRC cells transfected with pW24. Cell lysates from transfected cells were analyzed on a 12% SDS-containing, denaturing polyacrylamide gel, transferred to NC membrane, and probed with OP-1 antibody and 2nd Ab-HRP conjugate. Signals were developed with an ECL kit. Lane 1: No DNA control. Lane 2: pCMV control DNA. Lane 3: pCMV plus pA control DNAs. Lane 4: pW24 (1 mg/ml). Lane 5: pW24 plus pI (0.5 μg/ml). (B) AP activity in pW24 transfected FRC cells. Total AP activity in transfected cell lysates was measured after 24 (u) and 48 (n) h post-transfection. Values represent the means of two independent determinations using two different FRC cell preparations. Each determination involved 6 replicate samples.

FIG. 2 shows bone nodule formation in FRC cells transfected with pW24. Confluence FRC cells in 6-well plates were transfected with pW24 using FuGene6. Cells were cultured in complete αMEM containing 5% FBS, ascorbic acid, β-glycerol phosphate, and Neomycin. Media were changed every 3 days. Progress of nodule formation was monitored periodically and the images were captured 26 and 32 days after transfection using an Olympus CK2 inverted microscope.

FIG. 3A shows the effect of exogenous OP-1 and IGF-I on mock-transfected cells. Cells were mock transfected and treated for 48 h with OP-1 (200 ng/ml) alone or OP-1 (200 ng/ml) plus IGF-I (25 ng/ml) . Total AP activity in cell lysates were determined. Values were normalized to the control (=1). FIG. 3B shows the effect of exogenous IGF-I on AP activity in pW24 transfected FRC cells. FRC cells were transfected first with of 2 μg/ml pW24 and then cultured in complete MEM media in the presence of varying concentrations (0-37.5 ng/ml) of IGF-I. Total AP activity was measured after 48 h. Values were normalized to the pW24 transfected sample (=1) and represent the means of two independent determinations using two different FRC cell preparations. Each determination involved 6 replicate samples.

FIG. 4 shows the total AP activity in FRC cells co-transfected with plasmids pW24 and pI. Confluent FRC cells were co-transfected with 5 μg/ml of pW24 and varying concentrations of pI (0, 1, 2, 5, 10, and 20 μg/ml). After 48 h of recovery in complete media, total AP activity was measured. Values were normalized to the pW24 transfected (=1) and represent the mean±SEM of seven independent determinations with 3 separate FRC cell preparations and 3 different DNA preparations. pA is an empty vector without the IGF-I or the OP-1 gene.

FIG. 5 shows that the effect of IL-6+IL-6 receptor on OP-1-induced AP activity in FRC cells.

FIG. 6 shows the effect of IL-6 receptor on OP-1-induced AP activity in FRC cells.

FIG. 7 is a map of pW24. Human OP-1 (SmaI and BamHI fragment, 1.36 kb) was cloned into the pCMV/Neo vector (˜9 kb). It contains the full-length human OP-1 gene (from −23 to +1337, +1 is the translation start site, GenBank Accession # X51801).

FIG. 8 shows the human OP-1 nucleotide and protein sequences. The pro-peptide and mature protein domains are identified.

FIG. 9 is a map of the IGF-1 construct. The human IGF-I cDNA clone was originally obtained from ATCC (IMAGE Clone ID 502856). The human IGF-I gene was cloned in pT7T3D-Pac vector. Human IGF-I (EcoRI/DraI fragment, 0.74 kb) was subcloned into pcDNA4/TO/myc-HisA Vector (5.1 kb, from Invitrogen) at EcoRI and EcoRV sites for expression. It contains the full length human IGF-I gene (from −159 to +574, +1 is the translation start site, GenBank Accession # AA128355).

FIG. 10 shows the human IGF-1 nucleotide and protein sequences. The mature protein is identified.

### DETAILED DESCRIPTION OF THE INVENTION

In order that the invention herein described may be fully understood, the following detailed description is set forth.

Unless defined otherwise, all technical and scientific terms used herein have the same meaning as those commonly understood by one of ordinary skill in the art to which this invention belongs. Although methods and materials similar or equivalent to those described herein can be used in the practice or testing of the present invention, suitable methods and materials are described below. The materials, methods and examples are illustrative only, and are not intended to be limiting. All publications, patents and other documents mentioned herein are incorporated by reference in their entirety.

Throughout this specification, the word “comprise” or variations such as “comprises” or “comprising” will be understood to imply the inclusion of a stated integer or groups of integers but not the exclusion of any other integer or group of integers.

In order to further define the invention, the following terms and definitions are provided herein.

The term “morphogenic protein” refers to a protein having morphogenic activity (see below). Preferably a morphogenic protein of this invention comprises at least one polypeptide belonging to the BMP protein family. Morphogenic proteins may be capable of inducing progenitor cells to proliferate and/or to initiate differentiation pathways that lead to cartilage, bone, tendon, ligament, neural or other types of tissue formation depending on local environmental cues, and thus morphogenic proteins may behave differently in different surroundings. For example, an osteogenic protein may induce bone tissue at one treatment site and neural tissue at a different treatment site.

The term “bone morphogenic protein (BMP)” refers to a protein belonging to the BMP family of the TGF-β superfamily of proteins (BMP family) based on DNA and amino acid sequence homology. A protein belongs to the BMP family according to this invention when it has at least 50% amino acid sequence identity with at least one known BMP family member within the conserved C-terminal cysteine-rich domain, which characterizes the BMP protein family. Preferably, the protein has at least 70% amino acid sequence identity with at least one known BMP family member within the conserved C-terminal cysteine rich domain. Members of the BMP family may have less than 50% DNA or amino acid sequence identity overall. Bone morphogenic proteins may be monomeric, homo- or hetero-dimeric. Bone morphogenic proteins include osteogenic proteins.

Bone morphogenic proteins are capable of inducing progenitor cells to proliferate and/or to initiate differentiation pathways that lead to cartilage, bone, tendon, ligament or other types of tissue formation depending on local environmental cues, and thus bone morphogenic proteins may behave differently in different surroundings. For example, a bone morphogenic protein may induce bone tissue at one treatment site and cartilage tissue at a different treatment site. Bone morphogenic proteins include full length proteins as well as fragments thereof.

The term “osteogenic protein (OP)” refers to a bone morphogenic protein that is capable of inducing a progenitor cell to form cartilage and/or bone. The bone may be intramembranous bone or endochondral bone. Most osteogenic proteins are members of the BMP protein family and are thus also BMPs. As described elsewhere herein, the class of proteins is typified by human osteogenic protein (hOP-1). Other osteogenic proteins useful in the practice of the invention include but are not limited to, osteogenically active forms of OP-1, OP-2, OP-3, COP-1, COP-3, COP-4, COP-5, COP-7, COP-16, BMP-2, BMP-3, BMP-3b, BMP-4, BMP-5, BMP-6, BMP-9, BMP-10, BMP-11, CDMP-3 (BMP-12), CDMP-2 (BMP-13), CDMP-1 (BMP-14), BMP-15, BMP-16, BMP-17, BMP-18, GDF-1, GDF-2, GDF-3, GDF-5, GDF-6, GDF-7, GDF-8, GDF-9, GDF-10, GDF-11, GDF-12, MP121, dorsalin-1, DPP, Vg-1, Vgr-1, 60A protein, NODAL, UNIVIN, SCREW, ADMP, NEURAL, conservative amino acid sequence variants thereof having osteogenic activity and fragments thereof. In one currently preferred embodiment, the osteogenic protein is OP-1, amino acid sequence variants and homologs thereof, including species homologs thereof and fragments thereof. Particularly preferred osteogenic proteins are those comprising an amino acid sequence having at least 70% homology with the C-terminal 102-106 amino acids, defining the conserved seven cysteine domain, of, e.g., human OP-1. Certain preferred embodiments of the instant invention comprise the osteogenic protein, OP-1. As further described elsewhere herein, the osteogenic proteins suitable for use with this invention can be identified by means of routine experimentation using the art-recognized bioassay described by Reddi and Sampath (Sampath et al., Proc. Natl. Acad. Sci., 84, pp. 7109-13, incorporated herein by reference).

Proteins useful in this invention include eukaryotic proteins identified as osteogenic proteins (see U.S. Pat. No. 5,011,691, incorporated herein by reference), such as the OP-1, OP-2, OP-3, BMP-2, and BMP-3 proteins, as well as amino acid sequence-related proteins, such as DPP (from Drosophila), Vg1 (from Xenopus), Vgr-1 (from mouse), GDF-1 (from humans, see Lee, PNAS, 88, pp. 4250-4254 (1991)), 60A (from Drosophila, see Wharton et al., PNAS, 88, pp. 9214-9218 (1991)), dorsalin-1 (from chick, see Basler et al., Cell, 73, pp. 687-702 (1993) and GenBank accession number L12032) and GDF-5 (from mouse, see Storm et al., Nature, 368, pp. 639-643 (1994)). The teachings of the above references are incorporated herein by reference. Additional useful proteins include biosynthetic morphogenic constructs disclosed in U.S. Pat. No. 5,011,691, incorporated herein by reference, e.g., COP-1, COP-3, COP-4, COP-5, COP-7 and COP-16, as well as other proteins known in the art. Still other proteins include osteogenically active forms of BMP-3b (see Takao, et al., Biochem. Biophys. Res. Comm., 219, pp. 656-662 (1996)). BMP-9 (see WO 95/33830), BMP-15 (see WO 96/35710), BMP-12 (see WO 95/16035), CDMP-1 (see WO 94/12814), CDMP-2 (see WO 94/12814), BMP-10 (see WO 94/26893), GDF-1 (see WO 92/00382), GDF-10 (see WO 95/10539), GDF-3 (see WO 94/15965) and GDF-7 (see WO 95/01802). The teachings of the above references are incorporated herein by reference. BMPs (identified by sequence homology) must have demonstrable osteogenic activity in a functional bioassay to be osteogenic proteins according to this invention.

The term “morphogenic protein stimulatory factor (MPSF)” refers to a factor that is capable of stimulating the ability of a morphogenic protein to induce tissue formation from a progenitor cell. The MPSF may have a direct or indirect effect on enhancing morphogenic protein inducing activity. For example, the MPSF may increase the bioactivity of another MPSF. Agents that increase MPSF bioactivity include, for example, those that increase the synthesis, half-life, reactivity with other biomolecules such as binding proteins and receptors, or the bioavailability of the MPSF.

The terms “morphogenic activity”, “inducing activity” and “tissue inductive activity” alternatively refer to the ability of an agent to stimulate a target cell to undergo one or more cell divisions (proliferation) that may optionally lead to cell differentiation. Such target cells are referred to generically herein as progenitor cells. Cell proliferation is typically characterized by changes in cell cycle regulation and may be detected by a number of means which include measuring DNA synthetic or cellular growth rates. Early stages of cell differentiation are typically characterized by changes in gene expression patterns relative to those of the progenitor cell, which may be indicative of a commitment towards a particular cell fate or cell type. Later stages of cell differentiation may be characterized by changes in gene expression patterns, cell physiology and morphology. Any reproducible change in gene expression, cell physiology or morphology may be used to assess the initiation and extent of cell differentiation induced by a morphogenic protein.

The term “amino acid sequence homology” is understood to include both amino acid sequence identity and similarity. Homologous sequences share identical and/or similar amino acid residues, where similar residues are conservative substitutions for, or “allowed point mutations” of, corresponding amino acid residues in an aligned reference sequence. Thus, a candidate polypeptide sequence that shares 70% amino acid homology with a reference sequence is one in which any 70% of the aligned residues are either identical to, or are conservative substitutions of, the corresponding residues in a reference sequence. Certain particularly preferred bone morphogenic polypeptides share at least 60%, and preferably 70% amino acid sequence identity with the C-terminal 102-106 amino acids, defining the conserved seven-cysteine domain of human OP-1 and related proteins.

Amino acid sequence homology can be determined by methods well known in the art. For instance, to determine the percent homology of a candidate amino acid sequence to the sequence of the seven-cysteine domain, the two sequences are first aligned. The alignment can be made with, e.g., the dynamic programming algorithm described in Needleman et al., J. Mol. Biol., 48, pp. 443 (1970), and the Align Program, a commercial software package produced by DNAstar, Inc. The teachings by both sources are incorporated by reference herein. An initial alignment can be refined by comparison to a multi-sequence alignment of a family of related proteins. Once the alignment is made and refined, a percent homology score is calculated. The aligned amino acid residues of the two sequences are compared sequentially for their similarity to each other. Similarity factors include similar size, shape and electrical charge. One particularly preferred method of determining amino acid similarities is the PAM250 matrix described in Dayhoff et al., Atlas of Protein Sequence and Structure, 5, pp. 345-352 (1978 & Supp.), which is incorporated herein by reference. A similarity score is first calculated as the sum of the aligned pair wise amino acid similarity scores. Insertions and deletions are ignored for the purposes of percent homology and identity. Accordingly, gap penalties are not used in this calculation. The raw score is then normalized by dividing it by the geometric mean of the scores of the candidate sequence and the seven-cysteine domain. The geometric mean is the square root of the product of these scores. The normalized raw score is the percent homology.

The term “conservative substitutions” refers to residues that are physically or functionally similar to the corresponding reference residues. That is, a conservative substitution and its reference residue have similar size, shape, electric charge, chemical properties including the ability to form covalent or hydrogen bonds, or the like. Preferred conservative substitutions are those fulfilling the criteria defined for an accepted point mutation in Dayhoff et al., supra. Examples of conservative substitutions are substitutions within the following groups: (a) valine, glycine; (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine. The term “conservative variant” or “conservative variation” also includes the use of a substituting amino acid residue in place of an amino acid residue in a given parent amino acid sequence, where antibodies specific for the parent sequence are also specific for, i.e., “cross-react” or “immuno-react” with, the resulting substituted polypeptide sequence.

The term “fragment thereof” or “fragment” refers to a stretch of at least about 5 amino acid residues. In some embodiments, this term refers to a stretch of at least about 10 amino acid residues. In other embodiments, it refers to a stretch of at least about 15 to 20 amino acid residues. The fragments may be naturally derived or synthetically generated. To be active, any fragment must have sufficient length to display biological activity.

The term “defect” or “defect site,” refers to a disruption of the specified tissue. A defect can assume the configuration of a “void”, which is understood to mean a three-dimensional defect such as, for example, a gap, cavity, hole or other substantial disruption in the structural integrity of the tissue (e.g., bone, chondral, osteochondral, neural, ligament, tendon). Moreover, a defect can also be a detachment of the tendon or ligament from its point of attachment to bone, cartilage or muscle. In certain embodiments, the defect is such that it is incapable of endogenous or spontaneous repair. A defect can be the result of accident, disease, and/or surgical manipulation.

The term “target locus” refers to the site in any tissue where bone, cartilage, tendon, ligament or neural tissue regeneration is desired. The target locus may be, but need not be a defect site.

The term “repair” refers to new tissue formation which is sufficient to at least partially fill the void or structural discontinuity at the defect site. Repair does not, however, mean, or otherwise necessitate, a process of complete healing or a treatment, which is 100% effective at restoring a defect to its pre-defect physiological/structural/mechanical state.

The term “therapeutically effective amount” refers to an amount effective to repair, regenerate, promote, accelerate, prevent degradation, or form tissue.

The term “patient” refers to an animal, including a mammal (e.g., a human).

Methods Using Morphogenic Proteins and MPSFs

The present invention provides a method for inducing a progenitor cell to proliferate or differentiate comprising the steps of contacting a progenitor cell with a nucleic acid encoding a morphogenic protein and a nucleic acid encoding a morphogenic protein stimulatory factor (MPSF). In another embodiment, the invention provides a method for inducing a progenitor cell to proliferate or differentiate comprising the steps of: a) providing a vector comprising a nucleic acid encoding a morphogenic protein operably linked to an expression control sequence and a vector comprising a nucleic acid encoding a MPSF operably linked to an expression control sequence, and b) contacting said progenitor cell with said vectors.

In some embodiments, the invention provides gene therapy methods for inducing tissue formation, repairing a tissue defect or regenerating tissue at a target locus. In some embodiments, the invention provides a method for inducing tissue formation, repairing a tissue defect or regenerating tissue, at a target locus in a mammal, comprising the step of administering to the target locus a nucleic acid encoding a morphogenic protein and a nucleic acid encoding a MPSF. In other embodiments, the invention provides a method for inducing tissue formation, repairing a tissue defect or regenerating tissue, at a target locus in a mammal, comprising the steps of: a) providing a vector comprising a nucleic acid encoding a morphogenic protein operably linked to an expression control sequence and a vector comprising a nucleic acid encoding a MPSF operably linked to an expression control sequence, and b) administering to the target locus said vector. In yet other embodiments, the invention provides a method for inducing tissue formation, repairing a tissue defect or regenerating tissue, at a target locus in a mammal, comprising the steps of: a) providing a cultured host cell expressing a recombinant morphogenic protein and a recombinant MPSF, and b) administering to the target locus the host cell expressing the recombinant morphogenic protein and the recombinant MPSF.

In some embodiments, the invention provides a method of inducing tissue formation, repairing a tissue defect or regenerating tissue, by in vivo gene therapy, comprising the step of administering to target locus in a patient, a viral vector comprising a nucleotide sequence that encodes a morphogenic protein and a viral vector comprising a nucleotide sequence that encodes a MPSF so that the morphogenic protein and MPSF are expressed from the nucleotide sequence in the mammal in an amount sufficient to induce progenitor cells to proliferate or differentiate.

In some embodiments, the invention provides a method for improving the tissue inductive activity in a mammal of a morphogenic protein capable of inducing tissue formation at a target locus by coadministering an effective amount of MPSF, the method comprising administering to the target locus a nucleic acid encoding the morphogenic protein and a nucleic acid encoding the MPSF.

The invention also provides a method of improving the tissue inductive activity in a mammal of a morphogenic protein capable of inducing tissue formation at a target locus by coadministering an effective amount of a MPSF, the method comprising administering to the target locus a vector comprising a nucleic acid encoding the morphogenic protein operably linked to an expression control sequence and a vector comprising a nucleic acid encoding the MPSF operably linked to an expression control sequence.

The invention also provides a method for improving the tissue inductive activity in a mammal of a morphogenic protein capable of inducing tissue formation at a target locus by coadministering an effective amount of a MPSF, the method comprising administering to the target locus a cell comprising a vector comprising a nucleic acid encoding the morphogenic protein operably linked to an expression control sequence and a cell comprising a vector comprising a nucleic acid encoding the MPSF operably linked to an expression control sequence.

In some embodiments, the MPSF synergistically enhances the tissue inductive activity of the morphogenic protein.

Morphogenic Proteins

The morphogenic proteins of this invention are capable of stimulating a progenitor cell to undergo cell division and differentiation, and that inductive activity may be enhanced in the presence of a MPSF. Many mammalian morphogenic proteins have been described. Some fall within a class of products called “homeodomain proteins”, named for their homology to the drosophila homeobox genes involved in phenotypic expression and identity of body segments during embryogenesis. Other morphogenic proteins are classified as peptide growth factors, which have effects on cell proliferation, cell differentiation, or both.

Bone Morphogenic Protein (BMP) Family

The BMP family, named for its representative bone morphogenic/osteogenic protein family members, belongs to the TGF-β protein superfamily. Of the reported “BMPs” (BMP-1 to BMP-18), isolated primarily based on sequence homology, all but BMP-1 remain classified as members of the BMP family of morphogenic proteins (Ozkaynak et al., EMBO J., 9, pp. 2085-93 (1990)).

The BMP family includes other structurally-related members which are bone morphogenic proteins, including the drosophila decapentaplegic gene complex (DPP) products, the Vg1 product of Xenopus laevis and its murine homolog, Vgr-1 (see, e.g., Massagué, Annu. Rev. Cell Biol., 6, pp. 597-641 (1990), incorporated herein by reference).

The Drosophila DPP and Xenopus Vg-1 gene products are 50% identical to each other (and 35-40% identical to TGF-β). Both the Dpp and Vg-1 products are morphogenic proteins that participate in early patterning events during embryogenesis of their respective hosts. These products appear to be most closely related to mammalian bone morphogenetic proteins BMP-2 and BMP-4, whose C-terminal domains are 75% identical with that of Dpp.

The C-terminal domains of BMP-3, BMP-5, BMP-6, and OP-1 (BMP-7) are about 60% identical to that of BMP-2, and the C-terminal domains of BMP-6 and OP-1 are 87% identical. BMP-6 is likely the human homolog of the murine Vgr-1 (Lyons et al., Proc. Natl. Acad. Sci. U.S.A., 86, pp. 4554-59 (1989)); the two proteins are 92% identical overall at the amino acid sequence level (U.S. Pat. No. 5,459,047, incorporated herein by reference). BMP-6 is 58% identical to the Xenopus Vg-1 product.

The naturally occurring bone morphogenic proteins share substantial amino acid sequence homology in their C-terminal regions (domains). Typically, the above-mentioned naturally occurring osteogenic proteins are translated as a precursor, having an N-terminal signal peptide sequence typically less than about 30 residues, followed by a “pro” domain that is cleaved to yield the mature C-terminal domain of approximately 100-140 amino acids. The signal peptide is cleaved rapidly upon translation, at a cleavage site that can be predicted in a given sequence using the method of Von Heijne, Nucleic Acids Research, 14, pp. 4683-4691 (1986). The pro domain typically is about three times larger than the fully processed mature C-terminal domain.

Another characteristic of the BMP protein family members is their ability to dimerize. Several bone-derived osteogenic proteins (OPs) and BMPs are found as homo- and heterodimers in their active forms. The ability of OPs and BMPs to form heterodimers may confer additional or altered morphogenic inductive capabilities on bone morphogenic proteins. Heterodimers may exhibit qualitatively or quantitatively different binding affinities than homodimers for OP and BMP receptor molecules. Altered binding affinities may in turn lead to differential activation of receptors that mediate different signaling pathways, which may ultimately lead to different biological activities or outcomes. Altered binding affinities could also be manifested in a tissue or cell type-specific manner, thereby inducing only particular progenitor cell types to undergo proliferation and/or differentiation.

In one preferred embodiment of this invention, the BMPs independently comprise a pair of subunits disulfide bonded to produce a dimeric species, wherein at least one of the subunits comprises a recombinant peptide belonging to the BMP protein family. In another preferred embodiment of this invention, the BMPs independently comprise a pair of subunits that produce a dimeric species formed through non-covalent interactions, wherein at least one of the subunits comprises a recombinant peptide belonging to the BMP protein family. Non-covalent interactions include Van der Waals, hydrogen bond, hydrophobic and electrostatic interactions. The dimeric species may be a homodimer or heterodimer and is capable of inducing cell proliferation and/or tissue formation. In some embodiments, the BMPs are each independently monomers.

In preferred embodiments, the pair of morphogenic polypeptides have amino acid sequences each comprising a sequence that shares a defined relationship with an amino acid sequence of a reference morphogen. Herein, preferred osteogenic polypeptides share a defined relationship with a sequence present in osteogenically active human OP-1, SEQ ID NO: 1. However, any one or more of the naturally occurring or biosynthetic sequences disclosed herein similarly could be used as a reference sequence. Preferred osteogenic polypeptides share a defined relationship with at least the C-terminal six cysteine domain of human OP-1, residues 335-431 of SEQ ID NO: 1. Preferably, osteogenic polypeptides share a defined relationship with at least the C-terminal seven cysteine domain of human OP-1, residues 330-431 of SEQ ID NO: 1. That is, preferred polypeptides in a dimeric protein with bone morphogenic activity each comprise a sequence that corresponds to a reference sequence or is functionally equivalent thereto.

Functionally equivalent sequences include functionally equivalent arrangements of cysteine residues disposed within the reference sequence, including amino acid insertions or deletions which alter the linear arrangement of these cysteines, but do not materially impair their relationship in the folded structure of the dimeric morphogen protein, including their ability to form such intra- or inter-chain disulfide bonds as may be necessary for morphogenic activity. Functionally equivalent sequences further include those wherein one or more amino acid residues differs from the corresponding residue of a reference sequence, e.g., the C-terminal seven cysteine domain (also referred to herein as the conserved seven cysteine skeleton) of human OP-1, provided that this difference does not destroy bone morphogenic activity. Accordingly, conservative substitutions of corresponding amino acids in the reference sequence are preferred. Particularly preferred conservative substitutions are those fulfilling the criteria defined for an accepted point mutation in Dayhoff et al., supra, the teachings of which are incorporated by reference herein.

The osteogenic protein OP-1 has been described (see, e.g., Oppermann et al., U.S. Pat. No. 5,354,557, incorporated herein by reference). Natural-sourced osteogenic protein in its mature, native form is a glycosylated dimer typically having an apparent molecular weight of about 30-36 kDa as determined by SDS-PAGE. When reduced, the 30 kDa protein gives rise to two glycosylated peptide subunits having apparent molecular weights of about 16 kDa and 18 kDa. The unglycosylated protein, which also has osteogenic activity, has an apparent molecular weight of about 27 kDa. When reduced, the 27 kDa protein gives rise to two unglycosylated polypeptides, having molecular weights of about 14 kDa to 16 kDa, capable of inducing endochondral bone formation in a mammal. Osteogenic proteins may include forms having varying glycosylation patterns, varying N-termini, and active truncated or mutated forms of native protein.

As described above, particularly useful sequences include those comprising the C-terminal 96 or 102 amino acid sequences of DPP (from Drosophila), Vg1 (from Xenopus), Vgr-1 (from mouse), the OP-1 and OP-2 proteins, (see U.S. Pat. No. 5,011,691 and Oppermann et al., incorporated herein by reference), as well as the proteins referred to as BMP-2, BMP-3, BMP-4 (see WO 88/00205, U.S. Pat. No. 5,013,649 and WO 91/18098, incorporated herein by reference), BMP-5 and BMP-6 (see WO 90/11366, PCT/US90/01630, incorporated herein by reference), BMP-8 and BMP-9.

Preferred BMPs of this invention comprise at least one polypeptide selected from the group consisting of OP-1 (BMP-7), OP-2, OP-3, COP-1, COP-3, COP-4, COP-5, COP-7, COP-16, BMP-2, BMP-3, BMP-3b, BMP-4, BMP-5, BMP-6, BMP-9, BMP-10, BMP-11, CDMP-3 (BMP-12), CDMP-2 (BMP-13), CDMP-1 (BMP-14), BMP-15, BMP-16, BMP-17, BMP-18, GDF-1, GDF-2, GDF-3, GDF-5, GDF-6, GDF-7, GDF-8, GDF-9, GDF-10, GDF-11, GDF-12, MP121, dorsalin-1, DPP, Vg-1, Vgr-1, 60A protein, NODAL, UNIVIN, SCREW, ADMP, NEURAL and amino acid sequence variants and homologs thereof, including species homologs thereof and fragments thereof. In some embodiments, the preferred BMP is OP-1 (BMP-7) or a fragment thereof.

Publications disclosing these sequences, as well as their chemical and physical properties, include: OP-1 and OP-2 (U.S. Pat. No. 5,011,691; U.S. Pat. No. 5,266,683; Ozkaynak et al., EMBO J, 9, pp. 2085-2093 (1990); OP-3 (WO 94/10203 (PCT US93/10520)), BMP-2, BMP-3, BMP-4, (WO 88/00205; Wozney et al. Science, 242, pp. 1528-1534 (1988)), BMP-5 and BMP-6, (Celeste et al., PNAS, 87, 9843-9847 (1991)), Vgr-1 (Lyons et al., PNAS, 86, pp. 4554-4558 (1989)); DPP (Padgett et al., Nature, 325, pp. 81-84 (1987)); Vg-1 (Weeks, Cell, 51, pp. 861-867 (1987)); BMP-9 (WO 95/33830 (PCT/US95/07084); BMP-10 (WO 94/26893 (PCT/US94/05290); BMP-11 (WO 94/26892 (PCT/US94/05288); BMP-12 (WO95/16035 (PCT/US94/14030); BMP-13 (WO95/16035 (PCT/US94/14030); GDF-1 (WO 92/00382 (PCT/US91/04096) and Lee et al. PNAS, 88, pp. 4250-4254 (1991); GDF-8 (WO 94/21681 (PCT/US94/03019); GDF-9 (WO 94/15966 (PCT/US94/00685); GDF-10 (WO 95/10539 (PCT/US94/11440); GDF-11 (WO 96/01845 (PCT/US95/08543); BMP-15 (WO 96/36710 (PCT/US96/06540); MP-121 (WO 96/01316 (PCT/EP95/02552); GDF-5 (CDMP-1, MP52) (WO 94/15949 (PCT/US94/00657) and WO 96/14335 (PCT/US94/12814) and WO 93/16099 (PCT/EP93/00350)); GDF-6 (CDMP-2, BMP13) (WO 95/01801 (PCT/US94/07762) and WO 96/14335 and WO 95/10635 (PCT/US94/14030)); GDF-7 (CDMP-3, BMP12) (WO 95/10802 (PCT/US94/07799) and WO 95/10635 (PCT/US94/14030)). The above publications are incorporated herein by reference.

In another embodiment of this invention, the BMPs may be prepared synthetically. BMPs prepared synthetically may be native, or may be non-native proteins, i.e., those not otherwise found in nature. Non-native osteogenic proteins have been synthesized using a series of consensus DNA sequences (U.S. Pat. No. 5,324,819, incorporated herein by reference). These consensus sequences were designed based on partial amino acid sequence data obtained from natural osteogenic products and on their observed homologies with other genes reported in the literature having a presumed or demonstrated developmental function.

Several of the biosynthetic consensus sequences (called consensus osteogenic proteins or “COPs”) have been expressed as fusion proteins in prokaryotes. Purified fusion proteins may be cleaved, refolded, implanted in an established animal model and shown to have bone- and/or cartilage-inducing activity. The currently preferred synthetic osteogenic proteins comprise two synthetic amino acid sequences designated COP-5 (SEQ. ID NO: 2) and COP-7 (SEQ. ID NO: 3). Oppermann et al., U.S. Pat. Nos. 5,011,691 and 5,324,819, which are incorporated herein by reference, describe the amino acid sequences of COP-5 and COP-7 as shown below:

COP5 LYVDFS-DVGWDDWIVAPPGYQAFYCHGECPFPLAD
COP7 LYVDFS-DVGWNDWIVAPPGYHAFYCHGECPFPLAD
COP5 HFNSTN--H-AVVQTLVNSVNSKI--PKACCVPTELSA
COP7 HLNSTN--H-AVVQTLVNSVNSKI--PKACCVPTELSA
COP5 ISMLYLDENEKVVLKYNQEMVVEGCGCR
COP7 ISMLYLDENEKVVLKYNQEMVVEGCGCR

In these amino acid sequences, the dashes (-) are used as fillers only to line up comparable sequences in related proteins. Differences between the aligned amino acid sequences are highlighted.

The DNA and amino acid sequences of these and other BMP family members are published and may be used by those of skill in the art to determine whether a newly identified protein belongs to the BMP family.

In certain preferred embodiments, the BMPs useful herein independently include those in which the amino acid sequences comprise a sequence sharing at least 70% amino acid sequence homology or “similarity”, preferably 80%, more preferably 90%, even more preferably 95%, even more preferably 98% homology or similarity, with a reference bone morphogenic protein selected from the foregoing naturally occurring proteins. Preferably, the reference protein is human OP-1, and the reference sequence thereof is the C-terminal seven cysteine domain present in osteogenically active forms of human OP-1, residues 330-431 of SEQ ID NO: 1. In some embodiments, the BMP comprises a dimeric protein having an amino acid sequence having at least 70% homology within the C-terminal 102-106 amino acids of human OP-1. In certain embodiments, a polypeptide suspected of being functionally equivalent to a reference BMP polypeptide is aligned therewith using the method of Needleman, et al., supra, implemented conveniently by computer programs such as the Align program (DNAstar, Inc.). As noted above, internal gaps and amino acid insertions in the candidate sequence are ignored for purposes of calculating the defined relationship, conventionally expressed as a level of amino acid sequence homology or identity, between the candidate and reference sequences. In one preferred embodiment, the reference sequence is OP-1. Bone morphogenic proteins useful herein accordingly include allelic, phylogenetic counterpart and other variants of the preferred reference sequence, whether naturally-occurring or biosynthetically produced (e.g., including “muteins” or “mutant proteins”), as well as novel members of the general morphogenic family of proteins, including those set forth and identified above. Certain particularly preferred bone morphogenic polypeptides share at least 60% amino acid identity with the preferred reference sequence of human OP-1, still more preferably at least 65% amino acid identity therewith.

In another embodiment, useful BMPs include those sharing the conserved seven cysteine domain and sharing at least 70% amino acid sequence homology (similarity) within the C-terminal active domain, as defined herein. In still another embodiment, the BMPs of the invention can be defined as osteogenically active proteins having any one of the generic sequences defined herein, including OPX (SEQ ID NO: 4) and Generic Sequences 7 (SEQ ID NO: 5) and 8 (SEQ ID NO: 6), or Generic Sequences 9 (SEQ ID NO: 7) and 10 (SEQ ID NO: 8).

The family of bone morphogenic polypeptides useful in the present invention, and members thereof, can be defined by a generic amino acid sequence. For example, Generic Sequence 7 (SEQ ID NO: 5) and Generic Sequence 8 (SEQ ID NO: 6) are 97 and 102 amino acid sequences, respectively, and accommodate the homologies shared among preferred protein family members identified to date, including at least OP-1, OP-2, OP-3, CBMP-2A, CBMP-2B, BMP-3, 60A, DPP, Vg1, BMP-5, BMP-6, Vgr-1, and GDF-1. The amino acid sequences for these proteins are described herein and/or in the art, as summarized above. The generic sequences include both the amino acid identity shared by these sequences in the C-terminal domain, defined by the six and seven cysteine skeletons (Generic Sequences 7 and 8, respectively), as well as alternative residues for the variable positions within the sequence. The generic sequences provide an appropriate cysteine skeleton where inter- or intramolecular disulfide bonds can form, and contain certain critical amino acids likely to influence the tertiary structure of the folded proteins. In addition, the generic sequences allow for an additional cysteine at position 36 (Generic Sequence 7) or position 41 (Generic Sequence 8), thereby encompassing the morphogenically active sequences of OP-2 and OP-3.

Generic Sequence 7

```
                        Leu Xaa Xaa Xaa Phe Xaa Xaa
                        1 5
Xaa Gly Trp Xaa Xaa Xaa Xaa Xaa Xaa Pro
                        10 15
Xaa Xaa Xaa Xaa Ala Xaa Tyr Cys Xaa Gly
                        20 25
Xaa Cys Xaa Xaa Pro Xaa Xaa Xaa Xaa Xaa
                        30 35
Xaa Xaa Xaa Asn His Ala Xaa Xaa Xaa Xaa
                        40 45
Xaa Xaa Xaa Xaa Xaa Xaa Xaa Xaa Xaa Xaa
                        50 55
Xaa Xaa Xaa Cys Cys Xaa Pro Xaa Xaa Xaa
                        60 65
Xaa Xaa Xaa Xaa Xaa Leu Xaa Xaa Xaa Xaa
                        70 75
Xaa Xaa Xaa Val Xaa Leu Xaa Xaa Xaa Xaa
                        80 85
Xaa Met Xaa Val Xaa Xaa Cys Xaa Cys Xaa
                        90 95
```

wherein each Xaa independently is selected from a group of one or more specified amino acids defined as follows: “res.” means “residue” and Xaa at res.2=(Tyr or Lys); Xaa at res.3=Val or Ile); Xaa at res.4=(Ser, Asp or Glu); Xaa at res.6=(Arg, Gln, Ser, Lys or Ala); Xaa at res.7=(Asp or Glu); Xaa at res.8=(Leu, Val or Ile); Xaa at res.11=(Gln, Leu, Asp, His, Asn or Ser); Xaa at res.12=(Asp, Arg, Asn or Glu); Xaa at res.13=(Trp or Ser); Xaa at res.14=(Ile or Val); Xaa at res.15=(Ile or Val); Xaa at res.16 (Ala or Ser); Xaa at res.18=(Glu, Gln, Leu, Lys, Pro or Arg); Xaa at res.19=(Gly or Ser); Xaa at res.20=(Tyr or Phe); Xaa at res.21=(Ala, Ser, Asp, Met, His, Gln, Leu or Gly); Xaa at res.23=(Tyr, Asn or Phe); Xaa at res.26=(Glu, His, Tyr, Asp, Gln, Ala or Ser); Xaa at res.28=(Glu, Lys, Asp, Gln or Ala); Xaa at res.30=(Ala, Ser, Pro, Gln, Ile or Asn); Xaa at res.31=(Phe, Leu or Tyr); Xaa at res.33=(Leu, Val or Met); Xaa at res.34=(Asn, Asp, Ala, Thr or Pro); Xaa at res.35=(Ser, Asp, Glu, Leu, Ala or Lys); Xaa at res.36=(Tyr, Cys, His, Ser or Ile); Xaa at res.37=(Met, Phe, Gly or Leu); Xaa at res.38=(Asn, Ser or Lys); Xaa at res.39=(Ala, Ser, Gly or Pro); Xaa at res.40=(Thr, Leu or Ser); Xaa at res.44=(Ile, Val or Thr); Xaa at res.45=(Val, Leu, Met or Ile); Xaa at res.46=(Gln or Arg); Xaa at res.47=(Thr, Ala or Ser); Xaa at res.48=(Leu or Ile); Xaa at res.49=(Val or Met); Xaa at res.50=(His, Asn or Arg); Xaa at res.51=(Phe, Leu, Asn, Ser, Ala or Val); Xaa at res.52=(Ile, Met, Asn, Ala, Val, Gly or Leu); Xaa at res.53=(Asn, Lys, Ala, Glu, Gly or Phe); Xaa at res.54=(Pro, Ser or Val); Xaa at res.55=(Glu, Asp, Asn, Gly, Val, Pro or Lys); Xaa at res.56=(Thr, Ala, Val, Lys, Asp, Tyr, Ser, Gly, Ile or His); Xaa at res.57=(Val, Ala or Ile); Xaa at res.58=(Pro or Asp); Xaa at res.59=(Lys, Leu or Glu); Xaa at res.60=(Pro, Val or Ala); Xaa at res.63=(Ala or Val); Xaa at res.65=(Thr, Ala or Glu); Xaa at res.66=(Gln, Lys, Arg or Glu); Xaa at res.67=(Leu, Met or Val); Xaa at res.68=(Asn, Ser, Asp or Gly); Xaa at res.69=(Ala, Pro or Ser); Xaa at res.70=(Ile, Thr, Val or Leu); Xaa at res.71=(Ser, Ala or Pro); Xaa at res.72=(Val, Leu, Met or Ile); Xaa at res.74=(Tyr or Phe); Xaa at res.75=(Phe, Tyr, Leu or His); Xaa at res.76=(Asp, Asn or Leu); Xaa at res.77=(Asp, Glu, Asn, Arg or Ser); Xaa at res.78=(Ser, Gln, Asn, Tyr or Asp); Xaa at res.79=(Ser, Asn, Asp, Glu or Lys); Xaa at res.80=(Asn, Thr or Lys); Xaa at res.82=(Ile, Val or Asn); Xaa at res.84=(Lys or Arg); Xaa at res.85=(Lys, Asn, Gln, His, Arg or Val); Xaa at res.86=(Tyr, Glu or His); Xaa at res.87=(Arg, Gln, Glu or Pro); Xaa at res.88=(Asn, Glu, Trp or Asp); Xaa at res.90=(Val, Thr, Ala or Ile); Xaa at res.92=(Arg, Lys, Val, Asp, Gln or Glu); Xaa at res.93=(Ala, Gly, Glu or Ser); Xaa at res.95=(Gly or Ala) and Xaa at res.97=(His or Arg).

Generic Sequence 8 (SEQ ID NO: 6) includes all of Generic Sequence 7 and in addition includes the following sequence (SEQ ID NO: 9) at its N-terminus:

SEQ ID NO:9

Cys Xaa Xaa Xaa Xaa
1 5

Accordingly, beginning with residue 7, each “Xaa” in Generic Sequence 8 is a specified amino acid defined as for Generic Sequence 7, with the distinction that each residue number described for Generic Sequence 7 is shifted by five in Generic Sequence 8. Thus, “Xaa at res.2=(Tyr or Lys)” in Generic Sequence 7 refers to Xaa at res.7 in Generic Sequence 8. In Generic Sequence 8, Xaa at res.2=(Lys, Arg, Ala or Gln); Xaa at res.3=(Lys, Arg or Met); Xaa at res.4=(His, Arg or Gln); and Xaa at res.5=(Glu, Ser, His, Gly, Arg, Pro, Thr, or Tyr).

In another embodiment, useful osteogenic proteins include those defined by Generic Sequences 9 and 10, defined as follows.

Specifically, Generic Sequences 9 and 10 are composite amino acid sequences of the following proteins: human OP-1, human OP-2, human OP-3, human BMP-2, human BMP-3, human BMP-4, human BMP-5, human BMP-6, human BMP-8, human BMP-9, human BMP-10, human BMP-11, Drosophila 60A, Xenopus Vg-1, sea urchin UNIVIN, human CDMP-1 (mouse GDF-5), human CDMP-2 (mouse GDF-6, human BMP-13), human CDMP-3 (mouse GDF-7, human BMP-12), mouse GDF-3, human GDF-1, mouse GDF-1, chicken DORSALIN, dpp, Drosophila SCREW, mouse NODAL, mouse GDF-8, human GDF-8, mouse GDF-9, mouse GDF-10, human GDF-11, mouse GDF-11, human BMP-15, and rat BMP3b. Like Generic Sequence 7, Generic Sequence 9 is a 97 amino acid sequence that accommodates the C-terminal six cysteine skeleton and, like Generic Sequence 8, Generic Sequence 10 is a 102 amino acid sequence which accommodates the seven cysteine skeleton.

Generic Sequence 9

```
Xaa Xaa Xaa Xaa Xaa Xaa Xaa Xaa Xaa Xaa
1 5 10
Xaa Xaa Xaa Xaa Xaa Xaa Pro Xaa Xaa Xaa
                                15 20
Xaa Xaa Xaa Xaa Cys Xaa Gly Xaa Cys Xaa
                                25 30
Xaa Xaa Xaa Xaa Xaa Xaa Xaa Xaa Xaa Xaa
                                35 40
Xaa Xaa Xaa Xaa Xaa Xaa Xaa Xaa Xaa Xaa
                                45 50
Xaa Xaa Xaa Xaa Xaa Xaa Xaa Xaa Xaa Xaa
                                55 60
Xaa Cys Xaa Pro Xaa Xaa Xaa Xaa Xaa Xaa
                                65 70
Xaa Xaa Leu Xaa Xaa Xaa Xaa Xaa Xaa Xaa
                                75 80
Xaa Xaa Xaa Xaa Xaa Xaa Xaa Xaa Xaa Xaa
                                85 90
Xaa Xaa Xaa Cys Xaa Cys Xaa
                                95
```

wherein each Xaa is independently selected from a group of one or more specified amino acids defined as follows: “res.” means “residue” and Xaa at res. 1=(Phe, Leu or Glu); Xaa at res.2=(Tyr, Phe, His, Arg, Thr, Lys, Gln, Val or Glu); Xaa at res.3=(Val, Ile, Leu or Asp); Xaa at res.4=(Ser, Asp, Glu, Asn or Phe); Xaa at res.5=(Phe or Glu); Xaa at res.6=(Arg, Gln, Lys, Ser, Glu, Ala or Asn); Xaa at res.7=(Asp, Glu, Leu, Ala or Gln); Xaa at res.8=(Leu, Val, Met, Ile or Phe); Xaa at res.9=(Gly, His or Lys); Xaa at res.10=(Trp or Met); Xaa at res.11=(Gln, Leu, His, Glu, Asn, Asp, Ser or Gly); Xaa at res. 12=(Asp, Asn, Ser, Lys, Arg, Glu or His); Xaa at res.13=(Trp or Ser); Xaa at res.14=(Ile or Val); Xaa at res.15=(Ile or Val); Xaa at res.16=(Ala, Ser, Tyr or Trp); Xaa at res. 18=(Glu, Lys, Gln, Met, Pro, Leu, Arg, His or Lys); Xaa at res. 19=(Gly, Glu, Asp, Lys, Ser, Gln, Arg or Phe); Xaa at res.20=(Tyr or Phe); Xaa at res.21=(Ala, Ser, Gly, Met, Gln, His, Glu, Asp, Leu, Asn, Lys or Thr); Xaa at res.22=(Ala or Pro); Xaa at res.23=(Tyr, Phe, Asn, Ala or Arg); Xaa at res.24=(Tyr, His, Glu, Phe or Arg); Xaa at res.26=(Glu, Asp, Ala, Ser, Tyr, His, Lys, Arg, Gln or Gly); Xaa at res.28=(Glu, Asp, Leu, Val, Lys, Gly, Thr, Ala or Gln); Xaa at res.30=(Ala, Ser, Ile, Asn, Pro, Glu, Asp, Phe, Gln or Leu); Xaa at res.31=(Phe, Tyr, Leu, Asn, Gly or Arg); Xaa at res.32=(Pro, Ser, Ala or Val); Xaa at res.33=(Leu, Met, Glu, Phe or Val); Xaa at res.34=(Asn, Asp, Thr, Gly, Ala, Arg, Leu or Pro); Xaa at res.35(Ser, Ala, Glu, Asp, Thr, Leu, Lys, Gln or His); Xaa at res.36=(Tyr, His, Cys, Ile, Arg, Asp, Asn, Lys, Ser, Glu or Gly); Xaa at res.37=(Met, Leu, Phe, Val, Gly or Tyr); Xaa at res.38=(Asn, Glu, Thr, Pro, Lys, His, Gly, Met, Val or Arg); Xaa at res.39=(Ala, Ser, Gly, Pro or Phe); Xaa at res.40=(Thr, Ser, Leu, Pro, His or Met); Xaa at res.41=(Asn, Lys, Val, Thr or Gln); Xaa at res.42=(His, Tyr or Lys); Xaa at res.43=(Ala, Thr, Leu or Tyr); Xaa at res.44=(Ile, Thr, Val, Phe, Tyr, Met or Pro); Xaa at res.45=(Val, Leu, Met, Ile or His); Xaa at res.46=(Gln, Arg or Thr); Xaa at res.47=(Thr, Ser, Ala, Asn or His); Xaa at res.48=(Leu, Asn or Ile); Xaa at res.49=(Val, Met, Leu, Pro or Ile); Xaa at res.50=(His, Asn, Arg, Lys, Tyr or Gln); Xaa at res.51=(Phe, Leu, Ser, Asn, Met, Ala, Arg, Glu, Gly or Gln); Xaa at res.52=(Ile, Met, Leu, Val, Lys, Gln, Ala or Tyr); Xaa at res.53=(Asn, Phe, Lys, Glu, Asp, Ala, Gln, Gly, Leu or Val); Xaa at res.54=(Pro, Asn, Ser, Val or Asp); Xaa at res.55=(Glu, Asp, Asn, Lys, Arg, Ser, Gly, Thr, Gln, Pro or His); Xaa at res.56=(Thr, His, Tyr, Ala, Ile, Lys, Asp, Ser, Gly or Arg); Xaa at res.57=(Val, Ile, Thr, Ala, Leu or Ser); Xaa at res.58=(Pro, Gly, Ser, Asp or Ala); Xaa at res.59=(Lys, Leu, Pro, Ala, Ser, Glu, Arg or Gly); Xaa at res.60=(Pro, Ala, Val, Thr or Ser); Xaa at res.61=(Cys, Val or Ser); Xaa at res.63=(Ala, Val or Thr); Xaa at res.65=(Thr, Ala, Glu, Val, Gly, Asp or Tyr); Xaa at res.66=(Gln, Lys, Glu, Arg or Val); Xaa at res.67=(Leu, Met, Thr or Tyr); Xaa at res.68=(Asn, Ser, Gly, Thr, Asp, Glu, Lys or Val); Xaa at res.69=(Ala, Pro, Gly or Ser); Xaa at res.70=(Ile, Thr, Leu or Val); Xaa at res.71=(Ser, Pro, Ala, Thr, Asn or Gly); Xaa at res.72=(Val, Ile, Leu or Met); Xaa at res.74=(Tyr, Phe, Arg, Thr, Tyr or Met); Xaa at res.75=(Phe, Tyr, His, Leu, Ile, Lys, Gln or Val); Xaa at res.76=(Asp, Leu, Asn or Glu); Xaa at res.77=(Asp, Ser, Arg, Asn, Glu, Ala, Lys, Gly or Pro); Xaa at res.78=(Ser, Asn, Asp, Tyr, Ala, Gly, Gln, Met, Glu, Asn or Lys); Xaa at res.79=(Ser, Asn, Glu, Asp, Val, Lys, Gly, Gln or Arg); Xaa at res.80=(Asn, Lys, Thr, Pro, Val, Ile, Arg, Ser or Gln); Xaa at res.81=(Val, Ile, Thr or Ala); Xaa at res.82=(Ile, Asn, Val, Leu, Tyr, Asp or Ala); Xaa at res.83=(Leu, Tyr, Lys or Ile); Xaa at res.84=(Lys, Arg, Asn, Tyr, Phe, Thr, Glu or Gly); Xaa at res.85=(Lys, Arg, His, Gln, Asn, Glu or Val); Xaa at res.86=(Tyr, His, Glu or Ile); Xaa at res.87=(Arg, Glu, Gln, Pro or Lys); Xaa at res.88=(Asn, Asp, Ala, Glu, Gly or Lys); Xaa at res.89=(Met or Ala); Xaa at res.90=(Val, Ile, Ala, Thr, Ser or Lys); Xaa at res.91=(Val or Ala); Xaa at res.92=(Arg, Lys, Gln, Asp, Glu, Val, Ala, Ser or Thr); Xaa at res.93=(Ala, Ser, Glu, Gly, Arg or Thr); Xaa at res.95=(Gly, Ala or Thr); Xaa at res.97=(His, Arg, Gly, Leu or Ser). Further, after res.53 in rBMP3b and mGDF-10 there is an Ile; after res.54 in GDF-1 there is a T; after res.54 in BMP3 there is a V; after res.78 in BMP-8 and Dorsalin there is a G; after res.37 in hGDF-1 there is Pro, Gly, Gly, Pro.

Generic Sequence 10 (SEQ ID NO: 8) includes all of Generic Sequence 9 (SEQ ID NO: 7) and in addition includes the following sequence (SEQ ID NO: 9) at its N-terminus:

SEQ ID NO:9

Cys Xaa Xaa Xaa Xaa
1 5

Accordingly, beginning with residue 6, each “Xaa” in Generic Sequence 10 is a specified amino acid defined as for Generic Sequence 9, with the distinction that each residue number described for Generic Sequence 9 is shifted by five in Generic Sequence 10. Thus, “Xaa at res. 1=(Tyr, Phe, His, Arg, Thr, Lys, Gln, Val or Glu)” in Generic Sequence 9 refers to Xaa at res.6 in Generic Sequence 10. In Generic Sequence 10, Xaa at res.2=(Lys, Arg, Gln, Ser, His, Glu, Ala, or Cys); Xaa at res.3=(Lys, Arg, Met, Lys, Thr, Leu, Tyr, or Ala); Xaa at res.4=(His, Gln, Arg, Lys, Thr, Leu, Val, Pro, or Tyr); and Xaa at res.5=(Gln, Thr, His, Arg, Pro, Ser, Ala, Gln, Asn, Tyr, Lys, Asp, or Leu).

As noted above, certain currently preferred bone morphogenic polypeptide sequences useful in this invention have greater than 60% identity, preferably greater than 65% identity, with the amino acid sequence defining the preferred reference sequence of hOP-1. These particularly preferred sequences include allelic and phylogenetic counterpart variants of the OP-1 and OP-2 proteins, including the Drosophila 60A protein. Accordingly, in certain particularly preferred embodiments, useful BMPs include active proteins comprising pairs of polypeptide chains within the generic amino acid sequence herein referred to as “OPX” (SEQ ID NO: 4), which defines the seven cysteine skeleton and accommodates the homologies between several identified variants of OP-1 and OP-2. As described therein, each Xaa at a given position independently is selected from the residues occurring at the corresponding position in the C-terminal sequence of mouse or human OP-1 or OP-2.

```
SEQ ID NO:4
Cys Xaa Xaa His Glu Leu Tyr Val Ser Phe Xaa Asp Leu Gly Trp Xaa Asp Trp
1 5 10 15
Xaa Ile Ala Pro Xaa Gly Tyr Xaa Ala Tyr Tyr Cys Glu Gly Glu Cys Xaa Phe Pro
        20 25 30 35
Leu Xaa Ser Xaa Met Asn Ala Thr Asn His Ala Ile Xaa Gln Xaa Leu Val His Xaa
                40 45 50 55
Xaa Xaa Pro Xaa Xaa Val Pro Lys Xaa Cys Cys Ala Pro Thr Xaa Leu Xaa Ala
                        60 65 70
Xaa Ser Val Leu Tyr Xaa Asp Xaa Ser Xaa Asn Val Ile Leu Xaa Lys Xaa Arg
75 80 85 90
Asn Met Val Val Xaa Ala Cys Gly Cys His
                95 100
```

wherein Xaa at res.2=(Lys or Arg); Xaa at res.3=(Lys or Arg); Xaa at res.11=(Arg or Gln); Xaa at res.16=(Gln or Leu); Xaa at res.19=(Ile or Val); Xaa at res.23=(Glu or Gln); Xaa at res.26=(Ala or Ser); Xaa at res.35=(Ala or Ser); Xaa at res.39=(Asn or Asp); Xaa at res.41=(Tyr or Cys); Xaa at res.50=(Val or Leu); Xaa at res.52=(Ser or Thr); Xaa at res.56=(Phe or Leu); Xaa at res.57=(Ile or Met); Xaa at res.58=(Asn or Lys); Xaa at res.60=(Glu, Asp or Asn); Xaa at res.61=(Thr, Ala or Val); Xaa at res.65=(Pro or Ala); Xaa at res.71=(Gln or Lys); Xaa at res.73=(Asn or Ser); Xaa at res.75=(Ile or Thr); Xaa at res.80=(Phe or Tyr); Xaa at res.82=(Asp or Ser); Xaa at res.84=(Ser or Asn); Xaa at res.89=(Lys or Arg); Xaa at res.91=(Tyr or His); and Xaa at res.97=(Arg or Lys).

In still another preferred embodiment, useful BMPs have polypeptide chains with amino acid sequences comprising a sequence encoded by a nucleic acid that hybridizes, under low, medium or high stringency hybridization conditions, to DNA or RNA encoding reference BMP sequences, e.g., C-terminal sequences defining the conserved seven cysteine domains of OP-1, OP-2, BMP-2, BMP-4, BMP-5, BMP-6, 60A, GDF-5, GDF-6, GDF-7 and the like. As used herein, high stringent hybridization conditions are defined as hybridization according to known techniques in 40% formamide, 5×SSPE, 5X×Denhardt's Solution, and 0.1% SDS at 37° C. overnight, and washing in 0.1×SSPE, 0.1% SDS at 50° C. Standard stringent conditions are well characterized in commercially available, standard molecular cloning texts. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984): Nucleic Acid Hybridization (B. D. Hames & S. J. Higgins eds. 1984); and B. Perbal, A Practical Guide To Molecular Cloning (1984), the disclosures of which are incorporated herein by reference.

As noted above, proteins useful in the present invention generally are dimeric proteins comprising a folded pair of the above polypeptides. In some embodiments, the pair of polypeptides are not disulfide bonded. In some embodiments the pair of polypeptides are disulfide bonded. Such disulfide bonded BMPs are inactive when reduced, but are active as oxidized homodimers and when oxidized in combination with others of this invention to produce heterodimers. Thus, members of a folded pair of bone morphogenic polypeptides in a morphogenically active protein can be selected independently from any of the specific polypeptides mentioned above.

The BMPs encoded by the materials and methods of this invention include proteins comprising any of the polypeptide chains described above, and includes allelic and phylogenetic counterpart variants of these proteins, Accordingly, such active forms are considered the equivalent of the specifically described constructs disclosed herein. The proteins may include forms having varying glycosylation patterns, varying N-termini, a family of related proteins having regions of amino acid sequence homology, and active truncated or mutated forms of native or biosynthetic proteins, produced by expression of recombinant DNA in host cells.

The BMPs contemplated herein can be expressed from intact or truncated cDNA or from synthetic DNAs in prokaryotic or eukaryotic host cells, and purified, cleaved, refolded, and dimerized to form morphogenically active compositions. Alternatively, cells expressing recombinant may be used in the methods of this invention. Currently preferred host cells include, without limitation, prokaryotes including E. coli or eukaryotes including yeast, or mammalian cells, such as CHO, COS or BSC cells. One of ordinary skill in the art will appreciate that other host cells can be used to advantage. Detailed descriptions of the bone morphogenic proteins useful in the practice of this invention, including how to make, use and test them for osteogenic activity, are disclosed in numerous publications, including U.S. Pat. Nos. 5,266,683 and 5,011,691, the disclosures of which are incorporated by reference herein.

Thus, in view of this disclosure and the knowledge available in the art, skilled genetic engineers can isolate genes from cDNA or genomic libraries of various different biological species, which encode appropriate amino acid sequences, or construct DNAs from oligonucleotides, and then can express them in various types of host cells, including both prokaryotes and eukaryotes, to produce large quantities of active proteins.

Morphogenic Protein Stimulatory Factors (MPSF)

A morphogenic protein stimulatory factor (MPSF) according to this invention is a factor that is capable of stimulating the ability of a morphogenic protein to induce tissue formation from a progenitor cell. In one embodiment of this invention, a method for inducing a progenitor cell to proliferate or differentiate comprising the steps of contacting a progenitor cell with a nucleic acid encoding a morphogenic protein and a morphogenic protein stimulatory factor (MPSF) under conditions which are permissive for the uptake of the nucleic acids into the progenitor cell is provided.

In one embodiment of this invention, a method for inducing tissue formation at a target locus in a mammal comprising the step of administering to the target locus a nucleic acid encoding a morphogenic protein and a nucleic acid encoding a MPSF is provided. In another embodiment of this invention, a method for inducing tissue formation at target locus in a mammal, comprising administering to the target locus a vector comprising a nucleic acid encoding a morphogenic protein operably linked to an expression control sequence and a vector comprising a nucleic acid encoding a MPSF operably linked to an expression control sequence is provided. In yet another embodiment of this invention, a method for inducing tissue formation at target locus in a mammal comprising the step of administering to the target locus a cell comprising a vector comprising a nucleic acid encoding the morphogenic protein operably linked to an expression control sequence and a cell comprising a vector comprising a nucleic acid encoding a MPSF operably linked to an expression control sequence is provided.

One or more MPSFs are selected for use in concert with one or more morphogenic proteins according to the desired tissue type to be induced and the site at which the morphogenic protein and MPSF will be administered. The particular choice of a morphogenic protein(s)/MPSF(s) combination and the relative concentrations at which they are combined may be varied systematically to optimize the tissue type induced at a selected treatment site using the procedures described herein.

The preferred morphogenic protein stimulatory factors (MPSFS) of this invention are selected from the group consisting of hormones, cytokines and growth factors. Most preferred MPSFs for inducing bone and/or cartilage formation in concert with an osteogenic protein comprise at least one compound selected from the group consisting of insulin-like growth factor I (IGF-I), fibroblast growth factor (FGF), growth hormone (GH), insulin, parathyroid hormone (PTH), and interleukins (e.g., IL-6, IL-6 together with soluble IL-6 receptor (IL6/IL-6R)) (see, e.g., U.S. Pat. No. 6,696,410 for description of IL-6 and soluble IL-6 receptor).

Production or Expression of Morphogenic Proteins and MPSFs

The morphogenic proteins and MPSFs according to this invention may be produced by expressing an appropriate recombinant DNA molecule in a host cell.

In some embodiments of this invention, the morphogenic proteins and MPSFs and are produced by the expression of an appropriate recombinant DNA molecule in a host cell. The DNA and amino acid sequences of morphogenic proteins and MPSFs have been reported, and methods for their recombinant production are published and otherwise known to those of skill in the art. For a general discussion of cloning and recombinant DNA technology, see Ausubel et al., supra; see also Watson et al., Recombinant DNA, 2d ed. 1992 (W.H. Freeman and Co., New York).

For cloning and expressing morphogenic proteins and MPSFs, standard recombinant DNA techniques may be used. With the DNA sequence available, a DNA fragment encoding any of these proteins be inserted into an expression vector selected to work in conjunction with a desired host expression system. The DNA fragment is cloned into the vector with the proper transcription control elements. In some embodiments, the expression of the desired protein may be constitutive. In some embodiments, the expression of the desired protein is under the control of an inducible promoter.

Vectors

In some embodiments, the invention provides vectors comprising the nucleic acids encoding morphogenic proteins and MPSFs. The choice of vector and expression control sequences to which the nucleic acids of this invention are operably linked depends on the functional properties desired, e.g., protein expression, and the host cell to be transformed.

Expression control elements useful for regulating the expression of an operably linked coding sequence are known in the art. Examples include, but are not limited to, inducible promoters, constitutive promoters, secretion signals, and other regulatory elements. When an inducible promoter is used, it can be controlled, e.g., by a change in nutrient status (e.g. concentration of growth factors or BMPs), or a change in temperature, in the host cell medium.

An appropriate vector is selected according to the host system selected. Useful vectors include but are not limited to plasmids, cosmids, bacteriophage, insect and animal viral vectors, including retroviruses, and other single and double-stranded DNA viruses.

In some embodiments, it may be preferable to recombinantly produce a mammalian protein for therapeutic uses in mammalian cell culture systems in order to produce a protein whose structure resembles more closely that of the natural material. Recombinant protein production in mammalian cells requires the establishment of appropriate cells and cell lines that are easy to transfect, are capable of stably maintaining foreign DNA with an unrearranged sequence, and which have the necessary cellular components for efficient transcription, translation, post-translational modification and secretion of the protein. In addition, a suitable vector carrying the gene of interest is necessary.

DNA vector design for transfection into mammalian cells should include appropriate sequences to promote expression of the gene of interest, including: appropriate transcription initiation, termination and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion.

Preferred DNA vectors also include a marker gene and means for amplifying the copy number of the gene of interest. DNA vectors may also comprise stabilizing sequences (e.g., ori- or ARS-like sequences and telomere-like sequences), or may alternatively be designed to favor directed or non-directed integration into the host cell genome.

Substantial progress in the development of mammalian cell expression systems has been made and many aspects of the system are well characterized. A detailed review of the production of foreign proteins in mammalian cells, including useful cells, protein expression-promoting sequences, marker genes, and gene amplification methods, is disclosed in M. M. Bendig, Genetic Engineering, 7, pp. 91-127 (1988).

Particular details of the transfection, expression and purification of recombinant proteins are well documented and are understood by those of skill in the art. Further details on the various technical aspects of each of the steps used in recombinant production of foreign genes in mammalian cell expression systems can be found in a number of texts and laboratory manuals in the art. See, e.g., F. M. Ausubel et al., ed., Current Protocols in Molecular Biology, John Wiley & Sons, New York (1989).

Briefly, among the best characterized transcription promoters useful for expressing a foreign gene in a particular mammalian cell are the SV40 early promoter, the adenovirus major late promoter (AdMLP), the mouse metallothionein-I promoter (mMT-I), the Rous sarcoma virus (RSV) long terminal repeat (LTR), the mouse mammary tumor virus long terminal repeat (MMTV-LTR), and the human cytomegalovirus major intermediate-early promoter (hCMV). The DNA sequences for all of these promoters are known in the art and are available commercially.

One method of gene amplification in mammalian cell systems is the use of the selectable dihydrofolate reductase (DHFR) gene in a dhfr- cell line. Generally, the DHFR gene is provided on the vector carrying the gene of interest, and addition of increasing concentrations of the cytotoxic drug methotrexate (MTX) leads to amplification of the DHFR gene copy number, as well as that of the physically-associated gene of interest. DHFR as a selectable, amplifiable marker gene in transfected chinese hamster ovary cell lines (CHO cells) is particularly well characterized in the art. Other useful amplifiable marker genes include the adenosine deaminase (ADA) and glutamine synthetase (GS) genes.

In one expression system, gene amplification is further enhanced by modifying marker gene expression regulatory sequences (e.g., enhancer, promoter, and transcription or translation initiation sequences) to reduce the levels of marker protein produced. Lowering the level of DHFR transcription increases the DHFR gene copy number (and the physically-associated gene) to enable the transfected cell to adapt to growth in even low levels of methotrexate (e.g., 0.1 μM MTX). As will be appreciated by those skilled in the art, other useful weak promoters, different from those disclosed and preferred herein, can be constructed using standard vector construction methodologies. In addition, other, different regulatory sequences also can be modified to achieve the same effect.

Another gene amplification scheme relies on the temperature sensitivity (ts) of BSC40-tsA58 cells transfected with an SV40 vector. Temperature reduction to 33° C. stabilizes the temperature sensitive SV40 T antigen, which leads to the excision and amplification of the integrated transfected vector DNA thereby amplifying the physically associated gene of interest.

Eukaryotic cell expression vectors are known in the art and are commercially available. Typically, such vectors contain convenient restriction sites for insertion of the desired DNA segment.

Eukaryotic cell expression vectors may include a selectable marker, e.g., a drug resistance gene. The neomycin phosphotransferase (neo) gene (Southern et al., 1982, J. Mol. Anal. Genet. 1:327-341) is an example of such a gene.

To express the desired proteins of this invention, DNAs encoding the proteins (BMPs, MPSFs) are inserted into expression vectors such as plasmids, retroviruses, cosmids, YACs, EBV-derived episomes, and the like. The expression vector and expression control sequences are chosen to be compatible with the expression host cell used. In some embodiments, morphogenic proteins and MPSFs nucleic acids are inserted into separate vectors. In some embodiments, the morphogenic proteins and MPSFs nucleic acids are inserted into the same vector.

A convenient vector is one that encodes a functionally complete protein. To the extent secretion of a desired protein is required, the recombinant expression vector can also encode a signal peptide that facilitates secretion of the desired protein from a host cell.

Nucleic acid molecules encoding morphogenic proteins and MPSFs, and vectors comprising these nucleic acid molecules, can be used for transformation of a suitable host cell. Transformation can be by any suitable method. Methods for introduction of exogenous DNA into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors.

Transformation of host cells can be accomplished by conventional methods suited to the vector and host cell employed. For transformation of prokaryotic host cells, electroporation and salt treatment methods can be employed (Cohen et al., 1972, Proc. Natl. Acad. Sci. USA 69:2110-2114). For transformation of vertebrate cells, electroporation, cationic lipid or salt treatment methods can be employed. See, e.g., Graham et al., 1973, Virology 52:456-467; Wigler et al., 1979, Proc. Natl. Acad. Sci. USA 76:1373-1376f.

Host Cells

Host cells can be prokaryotic or eukaryotic. Useful host cells include but are not limited to bacteria such as E. coli, yeasts such as Saccharomyces and Picia, insect-baculovirus cell system, and primary, transformed or immortalized eukaryotic cells in culture. Preferred eukaryotic host cells include, but are not limited to, yeast and mammalian cells, e.g., Chinese hamster ovary (CHO) cell, NIH Swiss mouse embryo cells NIH-3T3, baby hamster kidney cells (BHK), C2C12 cells and BSC cells. Other useful eukaryotic cells include osteoprogenitor cells, cartilage progenitor cells, tendon progenitor cells, ligament progenitor cells and neural progenitor cells.

The methodology disclosed herein includes the use of COS cells for the rapid evaluation of vector construction and gene expression, and the use of established cell lines for long term protein production.

The choice of cells/cell lines is also important and depends on the needs of the skilled practitioner. Monkey kidney cells (COS) provide high levels of transient gene expression providing a useful means for rapidly testing vector construction and the expression of cloned genes. COS cells are transfected with a simian virus 40 (SV40) vector carrying the gene of interest. The transfected COS cells eventually die, thus preventing the long term production of the desired protein product. However, transient expression does not require the time consuming process required for the development of stable cell lines.

CHO cells are capable of successfully expressing a wide variety of proteins from a broad range of cell types. Thus, while the glycosylation pattern on a recombinant protein produced in a mammalian cell expression system may not be identical to the natural protein, the differences in oligosaccharide side chains are often not essential for biological activity of the expressed protein.

The DHFR gene also may be used as part of a gene amplification scheme for CHO cells. Another gene amplification scheme relies on the temperature sensitivity (ts) of BSC40-tsA58 cells transfected with an SV40 vector. Temperature reduction to 33° C. stabilizes the ts SV40 T antigen which leads to the excision and amplification of the integrated transfected vector DNA, thereby also amplifying the associated gene of interest.

Stable cell lines were established for CHO cells as well as BSC40-tsA58 cells (hereinafter referred to as “BSC cells”). The various cells, cell lines and DNA sequences chosen for mammalian cell expression of the BMPs and MPSFs of this invention are well characterized in the art and are readily available. Other promoters, selectable markers, gene amplification methods and cells also may be used to express the BMPs and MPSFs of this invention. Particular details of the transfection, expression, and purification of recombinant proteins are well documented in the art and are understood by those having ordinary skill in the art. Further details on the various technical aspects of each of the steps used in recombinant production of foreign genes in mammalian cell expression systems can be found in a number of texts and laboratory manuals in the art. See, e.g., F. M. Ausubel et al., ed., Current Protocols in Molecular Biology, John Wiley & Sons, New York (1989).

Progenitor Cells

The progenitor cells that are induced to proliferate and/or differentiate in the present invention are preferably mammalian cells. Preferred progenitor cells include mammalian chondroblasts, osteoblasts and neuroblasts, all earlier developmental precursors thereof, and all cells that develop therefrom (e.g., chondroblasts, pre-chondroblasts and chondrocytes). However, any non-mammalian progenitor cells are also likely to be useful in the methods of the present invention.

In some embodiments, the progenitor cells comprise a nucleic acid encoding one or more morphogenic protein and a nucleic acid encoding one or more MPSF. In some embodiments, the nucleic acid encoding a morphogenic protein and the nucleic acid encoding a MPSF are in different cell types. In some embodiments, the nucleic acid encoding a morphogenic protein and the nucleic acid encoding a MPSF are in separate cells. In some embodiments, the progenitor cells comprise vectors comprising a nucleic acid encoding one or more morphogenic protein and a nucleic acid encoding one or more MPSF. In some embodiments, the nucleic acid encoding one or more morphogenic protein and a nucleic acid encoding one or more MPSF are in one vector. In some embodiments, the nucleic acid encoding one or more morphogenic protein and a nucleic acid encoding one or more MPSF are in separate vectors. In some embodiments, the nucleic acids are recombinant.

In some embodiments, more than one morphogenic protein-encoding nucleic acid will be administered to the desired cell or tissue. In some embodiments, two morphogenic protein-encoding nucleic acids will be used. In some embodiments, three morphogenic protein-encoding nucleic acids will be used. In some embodiments, more than one MPSF-encoding nucleic acid will be administered to the desired cell or tissue. In some embodiments, two MPSF-encoding nucleic acids will be used. In some embodiments, three MPSF-encoding nucleic acids will be used. The particular choice of combination of nucleic acids encoding morphogenic proteins and MPSFs and the relative concentrations at which they are combined may be varied systematically to optimize the tissue type induced at a selected treatment site using the procedures described herein. The relative concentrations of the nucleic acids encoding the morphogenic proteins and the MPSFs that will optimally induce tissue formation when administered to a mammal may be determined empirically by the skilled practitioner using the procedures described herein.

Gene Therapy

The morphogenic proteins and MPSFs can be produced in vivo in a mammal, e.g., a human patient, using a gene therapy approach for inducing tissue formation, repairing a tissue defect or regenerating tissue at a target locus. This involves administration of a suitable morphogenic protein- or MPSF-encoding nucleic acid operably linked to suitable expression control sequences. Preferably, these sequences are incorporated into a viral vector. Suitable viral vectors for such gene therapy include adenoviral vectors, lentiviral vectors, baculoviral vectors, Epstein Barr viral vectors, papovaviral vectors, vaccinia viral vectors, herpes simplex viral vectors, and adeno associated virus (AAV) vectors. The viral vector can be a replication-defective viral vector. A preferred adenoviral vector has a deletion in its E1 gene or E3 gene. When an adenoviral vector is used, preferably the mammal is not exposed to a nucleic acid encoding a selectable marker gene.

Pharmaceutical Compositions

The nucleic acids encoding the morphogenic proteins and MPSFs, vectors and cells comprising the nucleic acids according to the present invention can be formulated as part of a pharmaceutical composition. The compositions of this invention will be administered at an effective dose to induce the particular type of tissue at the treatment site selected according to the particular clinical condition addressed. Determination of a preferred pharmaceutical formulation and a therapeutically effective dose regimen for a given application is well within the skill of the art. A specific dosage and treatment regimen for any particular patient will depend upon a variety of factors, including the particular morphogenic protein and MPSF used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

Administration of the nucleic acids encoding the morphogenic proteins and MPSFs, vectors and cells comprising the nucleic acids of this invention, may be accomplished using any of the conventional modes of administration.

The pharmaceutical compositions comprising a nucleic acid encoding a morphogenic protein or MPSF, vector or cell comprising a nucleic acid of this invention may be in a variety of forms. These include, for example, solid, semi-solid and liquid dosage forms such as tablets, pills, powders, liquid solutions or suspensions, suppositories, and injectable and infusible solutions. The preferred form depends on the intended mode of administration and therapeutic application and may be selected by one skilled in the art. Modes of administration may include oral, parenteral, subcutaneous, intravenous, intralesional or topical administration. In most cases, the pharmaceutical compositions of this invention will be administered in the vicinity of the treatment site in need of tissue regeneration or repair.

The pharmaceutical compositions of this invention may, for example, be placed into sterile, isotonic formulations with or without cofactors which stimulate uptake or stability. The formulation is preferably liquid, or may be lyophilized powder.

Sterile injectable forms of the compositions used in the methods of this invention may be aqueous or oleaginous suspension. These suspensions may be formulated according to techniques known in the art using suitable dispersing or wetting agents and suspending agents. The sterile, injectable preparation may also be a sterile, injectable solution or suspension in a non-toxic parenterally acceptable diluent or solvent, for example as a suspension in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Parenteral formulations may be a single bolus dose, an infusion or a loading bolus dose followed with a maintenance dose. These compositions may be administered at specific fixed or variable intervals, e.g., once a day, or on an “as needed” basis.

The compositions also will preferably include conventional pharmaceutically acceptable carriers well known in the art (see for example Remington's Pharmaceutical Sciences, 16th Edition, 1980, Mac Publishing Company). The pharmaceutical compositions used in the methods of this invention comprise pharmaceutically acceptable carriers, including, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Such pharmaceutically acceptable carriers may include other medicinal agents, carriers, genetic carriers, adjuvants, excipients, etc., such as human serum albumin or plasma preparations. The compositions are preferably in the form of a unit dose and will usually be administered as a dose regimen that depends on the particular tissue treatment.

The pharmaceutical compositions of this invention may also be administered using, for example, microspheres, liposomes, other microparticulate delivery systems or sustained release formulations placed in, near, or otherwise in communication with affected tissues or the bloodstream bathing those tissues.

Liposomes containing a nucleic acid encoding a morphogenic proteins or MPSF, vector or cell comprising a nucleic acid of this invention can be prepared by well-known methods (See, e.g. DE 3,218,121; Epstein et al., Proc. Natl. Acad. Sci. U.S.A., 82, pp. 3688-92 (1985); Hwang et al., Proc. Natl. Acad. Sci. U.S.A., 77, pp. 4030-34 (1980); U.S. Pat. Nos. 4,485,045 and 4,544,545). Ordinarily the liposomes are of the small (about 200-800 Angstroms) unilamellar type in which the lipid content is greater than about 30 mol. % cholesterol. The proportion of cholesterol is selected to control the optimal rate of release of active agent.

The nucleic acids encoding morphogenic proteins and/or MPSFs, vectors or cells comprising the nucleic acids of this invention may also be attached to liposomes containing other biologically active molecules such as immunosuppressive agents, cytokines, etc., to modulate the rate and characteristics of tissue induction. Attachment to liposomes may be accomplished by any known cross-linking agent such as heterobifunctional cross-linking agents that have been widely used to couple toxins or chemotherapeutic agents to antibodies for targeted delivery. Conjugation to liposomes can also be accomplished using the carbohydrate-directed cross-linking reagent 4-(4-maleimidophenyl) butyric acid hydrazide (MPBH) (Duzgunes et al., J. Cell. Biochem. Abst. Suppl. 16E 77 (1992)).

The following are examples which illustrate the methods of this invention. These examples should not be construed as limiting: the examples are included for purposes of illustration and the present invention is limited only by the claims.

### EXAMPLE 1

### Construction of Plasmids

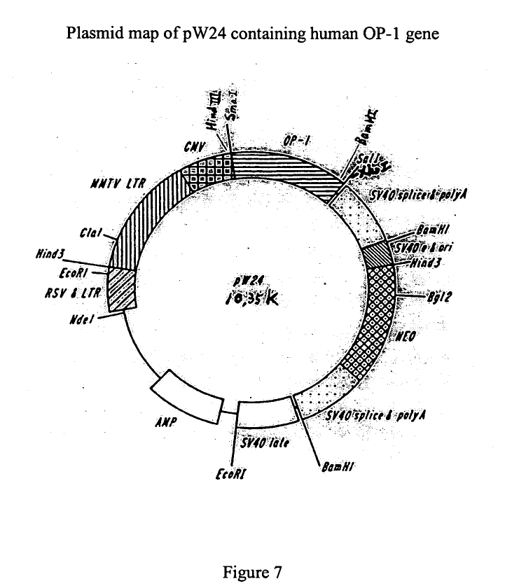

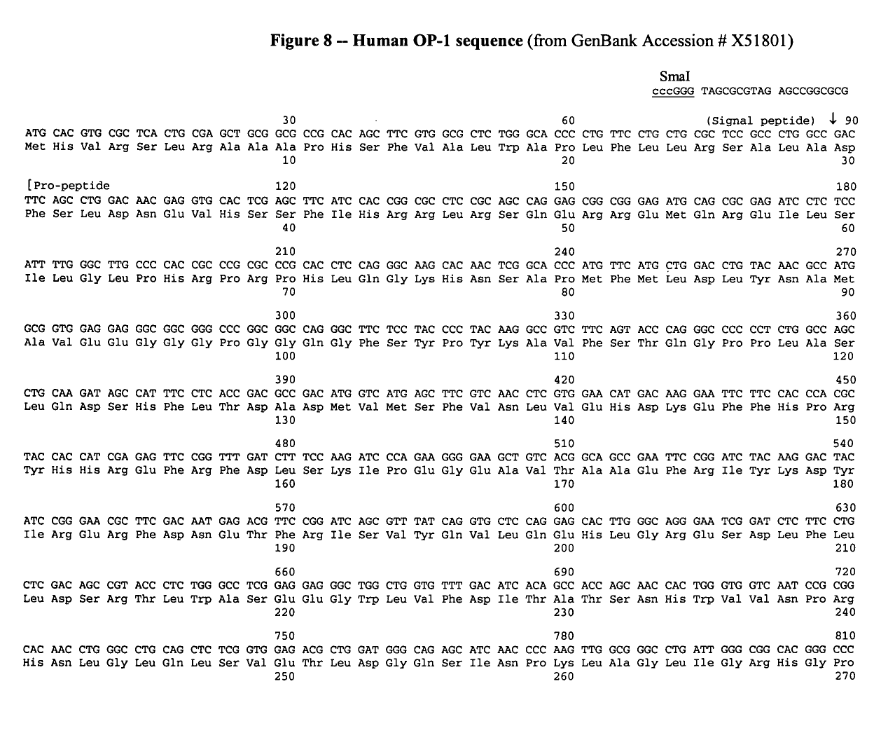

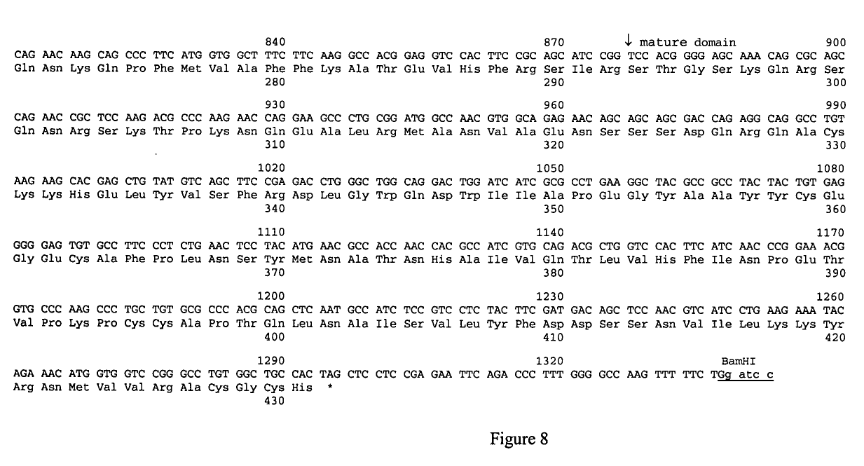

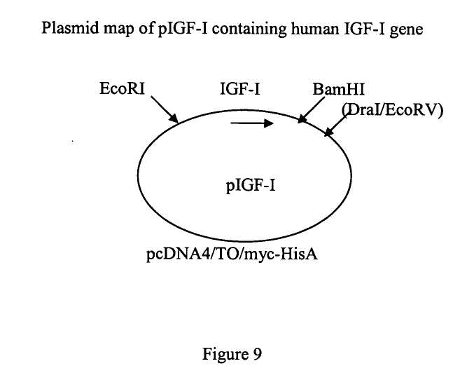

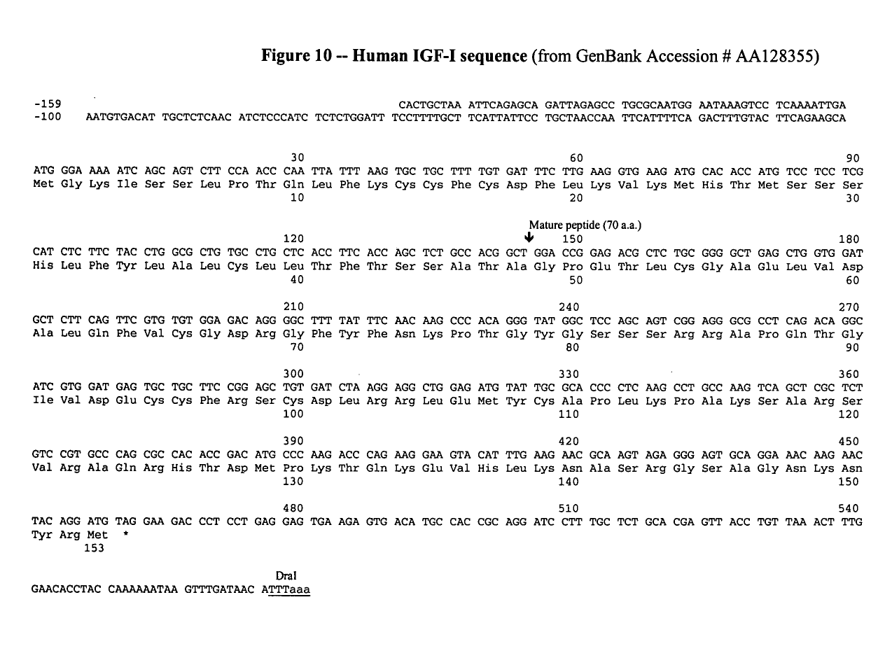

Plasmid pW24 (10.35 kb) contains the coding sequence for OP-1 under the control of the CMV promoter. To generate the control plasmid pCMV, pW24 was digested with restriction enzyme XhoI to remove the OP-1 coding sequence, purified on agarose gels, and re-ligated to produce the resultant 9.1 kb plasmid. See FIGS. 7 and 8. Plasmid pIGF-I (or pI, 5.88 kb) contains a 740 bp human IGF-I sequence inserted into the EcoRI/EcoRV site of pA (pcDNA4/TO/myc-HisA, 5.14 kb, Invitrogene, Carlsbad, Calif.). The IGF-I gene was under the control of the CMV promoter. The 740 bp IGF-I sequence was obtained by digestion of pT7T3D-Pac (IMAGE ID 502856 from ATCC, Manassas, Va.) with EcoRI/DraI. See FIGS. 9 and 10. The base sequence of all the resultant products was determined by double-strand sequencing.

### EXAMPLE 2

### Fetal Rat Calvaria Cell Culture and Transfection

Primary osteoblasts cell cultures were prepared by subjecting the calvarium from the fetus of timed pregnant Sprague-Dawley rats to sequential digestion with trypsin/collagenase as described previously L. C. Yeh, et al., Endocrinology 137 (1996) 1921-1931. Transfection studies were carried out using methods well known in the art such as the calcium phosphate-DNA co-precipitation method or FuGene6 (Roche, Indianapolis, Ind.). Transfected FRC cells were grown in complete αMEM containing 10% FBS. All plasmid DNAs were isolated using the Qiagen's plasmid Maxi kit (Valencia, Calif.) and checked for purity on 1% agarose gels. Only the ultrapure DNA prep was used for transfection.

### EXAMPLE 3

### Alkaline Phosphatase (AP) Activity Assay

After 24 or 48 h of treatment, cells grown in 48-well plates were rinsed with PBS and were lysed by sonication in 0.1% Trition X-100 in PBS (100 μl/well) for 5 min at room temperature. Total cellular AP activity was measured using a commercial assay kit (Sigma, St. Louis, Mo.) as described previously L. C. Yeh, et al., Endocrinology 137 (1996) 1921-1931.

### EXAMPLE 4

### Mineralized Bone Nodule Formation Assay

Formation of mineralized bone nodules in long-term FRC cultures was accessed as previously described in L. C. Yeh, et al., Endocrinology 138 (1997) 4181-4190. Transfected FRC cells in 6-well plates were cultured in αMEM containing 5% FBS, ascorbic acid (100 μg/ml), and 5 mM β-glycerolphosphate. Media were changed every 3 days. Progress of nodule formation was monitored using an Olympus CK2 inverted microscope (Olympus America, Inc., Melville, N.Y.) equipped with a CCD camera.

### EXAMPLE 5

### Western Blot Analysis

Total cellular proteins were resolved on a denaturing, SDS-containing polyacrylamide gel (12%). After electrophoresis, proteins were transferred onto a nitrocellulose membrane and probed with an anti-OP-1 polyclonal antibody. The antigen-antibody complex was detected with anti-rabbit IgG conjugated with HRP and the Supersignal ECL kit (Pierce, Rockford, Ill.) following the manufacturer's instruction.

### EXAMPLE 6

### Effect of Transfection with OP-1 Gene on OP-1 Protein Production

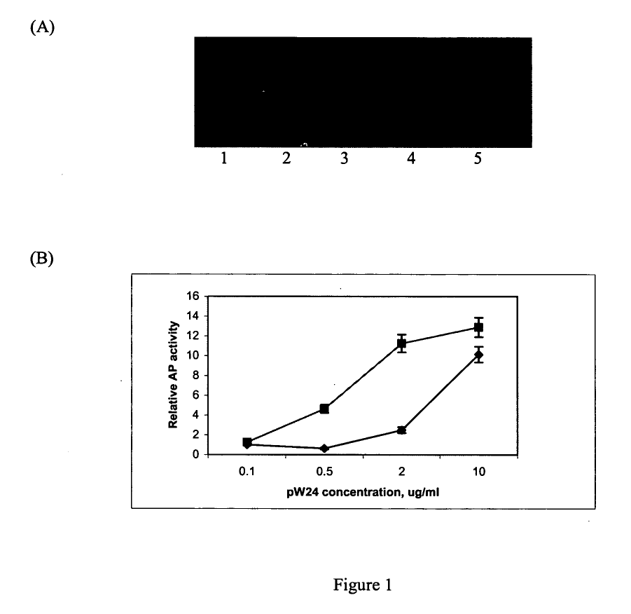

The OP-1 protein level was not detectable in Mock-transfected cells (FIG. 1A, lane 1), in cells transfected with the empty plasmid pCMV (lane 2), or with both empty plasmids pCMV and pA (lane 3). Upon transfection with pW24, a plasmid that contains the OP-1 coding sequence, the resultant cells expressed a protein with an approximate molecular weight of 16 kDa, a value consistent with the monomer of OP-1 and reacted with anti-OP-1 antibody by Western blot analysis (FIG. 1A, lane 4).

### EXAMPLE 7

### Effect of OP-1 Transfection on Cell Characteristics

FIG. 1B shows that cells transfected with pW24 exhibited a time- and DNA dose-dependent stimulation in AP activity. Cells transfected with 10 μg/ml pW24 showed a 10-fold increase in AP activity after 24 h. After 48 h, the AP activity in cells transfected with all the different concentrations of pW24 increased by 4- to 5.5-fold beyond those after 24 h, approaching a maximum stimulation about 13-fold with 10 /μg/ml pW24.

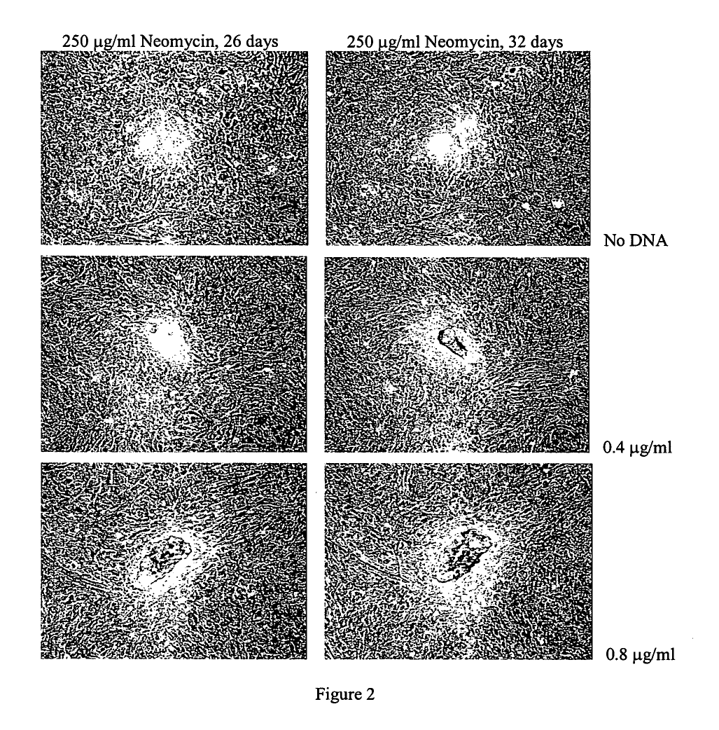

To examine long-term effects of pW24, cells were transfected with 0.4 or 0.8 μg/ml pW24 and cultured in the presence of 250 μg/ml neomycin. Bone nodule formation was observed in these cultures and was in a time- and DNA dose-dependent manner (FIG. 2). Clone 1 from the culture treated with 0.4 μg/ml and clone 2 from that with 0.8 μg/ml were selected for longer-term monitoring. After 26 days (FIG. 2, left column), the mock-transfected (top panel), clones 1 and 2 (middle and bottom, respectively) showed bone nodule formation, except that clone 2 showed mineralization. By 32 days (FIG. 2, right column), the nodules in the mock-transfected control remained about the same size and did not mineralized (top panel). The bone nodule of clone 1 became mineralized (middle panel). The size of the bone nodule and the extent of mineralization of clone 2 increased significantly (bottom panel). These results indicated that the transfected cell had undergone osteoblastic cell differentiation and that the OP-1 protein produced by the plasmid-coding OP-1 gene stimulated the differentiation process.

### EXAMPLE 8

### Effect of Exogenous IGF-I on AP Activity of FRC Cells Transfected with OP-1 Gene

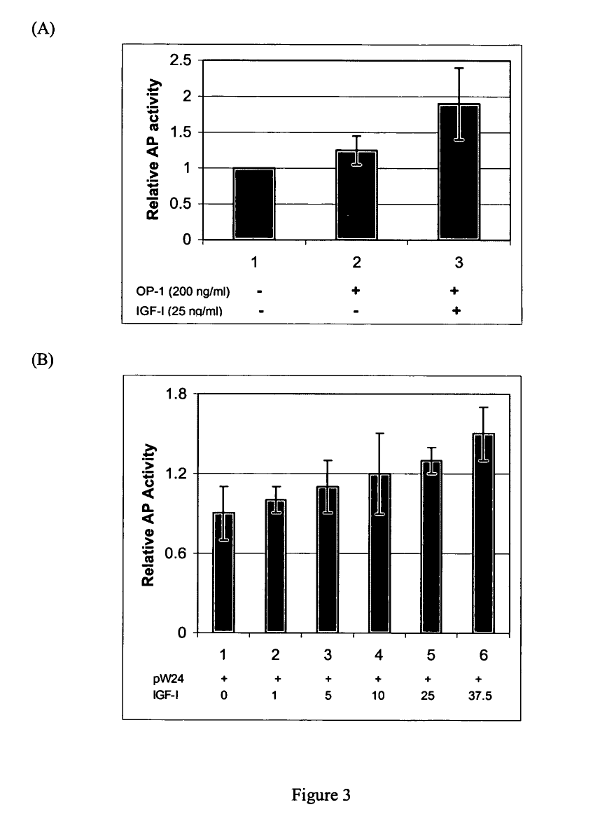

To test the effects of exogenous IGF-I on the pW24-transfected FRC cells, different concentrations of IGF-I were added to the media of the transfected cultures, and the total cellular AP activity was measured. To assess possible effects of the transfection procedure on the capability of these cells to respond to OP-1, the mock-transfected cells were treated with OP-1 or OP-1+IGF-I. OP-1 stimulated AP activity by about 1.4-fold above the vehicle control (FIG. 3A, lane 2 vs 1). Exogenous IGF-I further stimulated AP activity in the pW24-transfected cells by 1.8-fold above the control (FIG. 3A, compare lane 3 with lane 1). The extent of stimulation in AP activity in both cases was less than that usually observed with FRC cells not subjected to the transfection manipulation. A possible reason for the lower response may be that these mock-transfected FRC cells had not completely recovered from the shock of transfection. FIG. 3B shows that the relative AP activity in pW24-transfected cells treated with increasing concentrations of exogenous IGF-I was elevated in an IGF-I dose-dependent manner, reaching a maximum stimulation of 1.7-fold above that in cells transfected with pW24 alone (FIG. 3B, compare lanes 2 to 6 with lane 1).

### EXAMPLE 9

### Effect of OP-1 and IGF-I Gene Co-Transfection on AP Activity

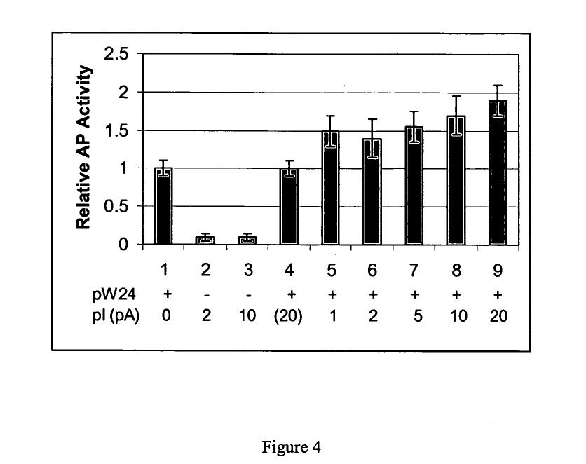

The effect of co-transfection of FRC cells with pW24 and pI, a plasmid containing the IGF-I gene under the control of the CMV promoter was examined. FRC cells were transfected with a constant amount of pW24 and increasing amounts of pI. After 48 h, the level of OP-1 protein expression and total AP activity were measured. FIG. 1A (lane 5) showed that the OP-1 expression levels in cells co-transfected with pW24 plus pI and with pW24 alone were similar. FIG. 4 shows that the AP activity in cells co-transfected with pW24 and pI increased as a function of pI concentration, reaching a maximum 2-fold stimulation beyond the OP-1-treated value and about 20-fold beyond the control (lanes 5- 9). The increase was beyond that in cells transfected with pW24 alone (lane 1). The AP activity in cells transfected with pI alone was not significant (lanes 2 and 3). Co-transfection with pW24 and the empty plasmid pA, (vector without the IGF-I gene) did not result in an increase in AP activity beyond that by pW24 alone (lane 4 vs 1). The observation implied that the stimulation of AP activity in the co-tranfected FRC cells was the result of the synergistic action of OP-1 and IGF-I proteins produced intracellularly. FRC cells co-transfected with the two empty plasmids, pCMV (vector without the OP-1 gene) and pA, did not show an increase in AP activity.

### EXAMPLE 10

### Effects of Exogenous IL-6 and Soluble IL-6 Receptor on AP Activity of FRC Cells Transfected with pW24

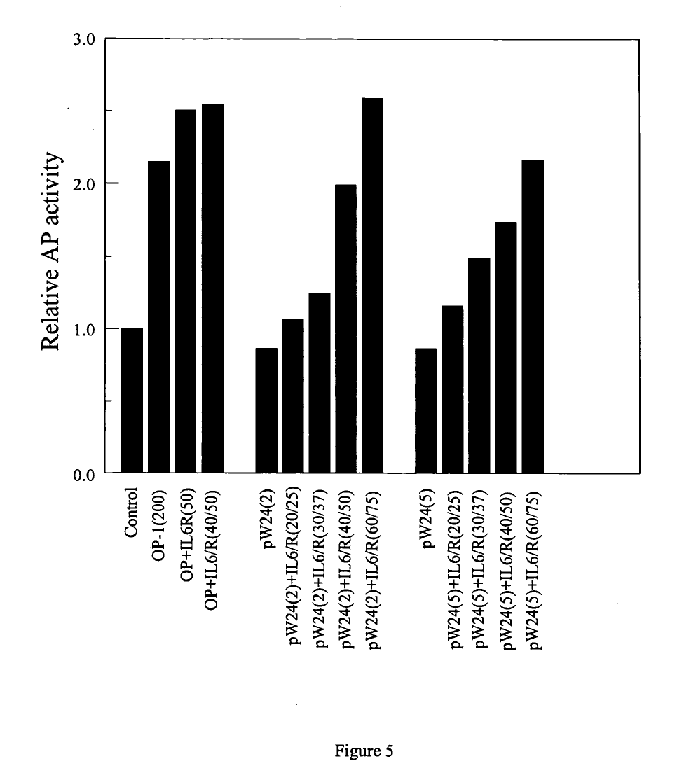

The effect of exogenous IL-6 or soluble IL-6 receptor (see e.g., U.S. Pat. No. 6,696,410) on the AP activity of FRC cells transfected with pW24 was tested. FIG. 5 shows that the levels of OP-1-induced AP activity in FRC cells transfected with pW24 (2 μg/ml) were enhanced in an IL-6 +soluble IL-6 receptor dose-dependent manner (FIG. 5, columns 5-9). At a dose of 60 ng/ml of IL-6 and 75 ng/ml of soluble IL-6 receptor, a 2.5-fold stimulation compared to the pW24-transfected value was observed (FIG. 5, column 9 vs column 5). The extent of the synergy increased with increasing concentrations of pW24 at the lower concentration range of IL-6+soluble IL-6 receptor (FIG. 5, columns 10-14 vs columns 5-9). However, at higher concentrations of IL-6+IL-6 receptors and a higher pW24 concentration (5 μg/ml), the synergy between IL-6+its soluble receptor and OP-1 was not as high (FIG. 5, columns 13-14 vs columns 8-9).

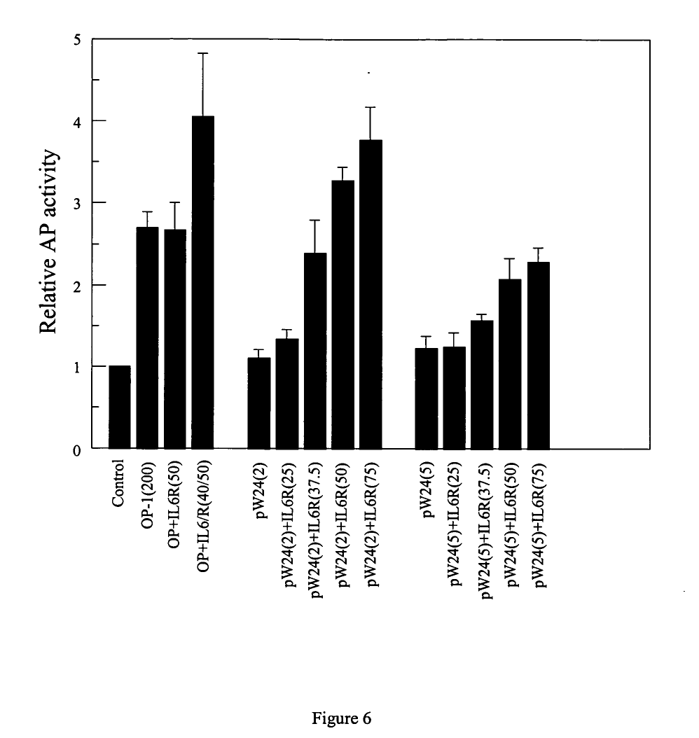

FIG. 6 shows that the levels of OP-1-induced AP activity in FRC cells transfected with pW24 (2 μg/ml) were enhanced in an IL-6 receptor dose-dependent manner (FIG. 6, columns 5-9). At a dose of 75 ng/ml of soluble IL-6 receptor, a 4-fold stimulation compared to the pW24-transfected value was observed (FIG. 6, column 9 vs column 5). However, when the FRC cells were transfected with a higher concentration of pW24 (5 μg/ml), the extent of the synergy was reduced (FIG. 6, columns 14-18 vs columns 5-9).

### EXAMPLE 11

### In vivo Expression of OP-1 with a MPSF

In vivo studies will be conducted using two experimental approaches: (i) Direct injection of OP-1 expressing vectors together with a MPSF (e.g., IGF-I, IGF-II, FGF, PTH, GH, insulin, IL-6 or IL-6/IL-6R) expressing vectors into muscles of mice, and (ii) injection of transfected cells into muscles.

For direct injection experiments, nude mice will be injected with vectors expressing OP-1 and a MPSF (e.g., IGF-I, IGF-II, FGF, PTH, GH, insulin, IL-6 or IL-6/IL-6R) with a 27-gauge needle subcutaneously into a male homozygous nude mouse. Standard aseptic techniques will be used in all manipulations. To determine in vivo osteogenic dose response of the vectors, eight mice will be used. Each mouse will be injected with 0.1-10 mg/ml vectors in 100 μl each. Body weight and growth at the site of injection will be followed daily via in-life measurement of the mass. The cross-sectional area of the mass will be measured with a vernier caliper. The size of the mass will be calculated using the formula: length/2×width/2×JI. The mass and the body weight will be plotted as a function of time following injection. The animals will be monitored for 49 days. At necropsy, the mass at the site of injection will be collected, fixed, stained with hematoxylin and eosin, and subjected to histological analysis. Controls will include mice injected with individual pW24 (OP-1), and pMPSF alone. It is anticipated that the bone mass in mice injected with the combination of the pW24 and pMPSF will be greater than that injected with individual vector alone.

For experiments using injection of cells, similar experiments as described above will be conducted except that animals will be injected with cells co-transfected with vectors carrying the OP-1, MPSF (e.g., IGF-I, IGF-II, FGF, PTH, GH, insulin, IL-6 or IL-6/IL-6R) genes. Accordingly, cells will be grown to mid-log phase and transfected with a combination of vectors expressing OP-1 and MPSF as described above using the optimal ratio of the two vectors. Cells will be removed from the culturing dishes by trypsin-EDTA digestion. Trypsin will be inactivated by serum (10%) and removed by repeated washings with HBSS. Cells will be suspended in a minimal volume of HBSS and injected with a 27-gauge needle subcutaneously into the flank of a male homozygous nude mouse. Standard aseptic techniques will be used in all manipulations. Eight nude mice will be injected with 10⁶ cells in 100 μl each. Outcome measurements as described above will be conducted. It is anticipated that the bone mass in mice injected with cells transfected with the combination of the pW24 and pMPSF will be greater than that injected with cells transfected with individual vector alone.

### EXAMPLE 12

### Gene Therapy in Patients Using Transfected Cells

For cell therapeutics with transfected genes, appropriate cells will be transfected in vitro with DNA vectors carrying the OP- I gene, the MPSF gene (e.g., IGF-I, IGF-II, FGF, PTH, GH, insulin, IL-6 or IL-6/IL-6R). Appropriate cells include osteoblasts or osteoblastic cell progenitors for the repair of bone defects. For repair of cartilage regeneration, cells of chondrocyte origin or chondrogenitor cells will be appropriate. Similarly, for the regeneration of tendons or ligaments, the appropriate cells include progenitor cells of tendon or ligament origin. The transfected cells will be cultured to allow expression of the transfected gene(s). The cells will then be injected or implanted into a defect site in a patient. The defect site may be in bone, cartilage, tendon, ligament or neural tissue. The number of cells injected or implanted into the defect will depend on the size of the defect. Exemplary DNA vectors will be pW24, pIGF-I, pIGF-II, pPTH, pInsulin, pGH, pFGF, pIL-6 or pIL-6/pIL-6R as described previously.

### EXAMPLE 13

### Gene Therapy in Patients Using Transfected Cells

For directed gene therapy, a combination of vectors as described above carrying the OP-1 gene, the IGF-I, IGF-II, FGF, PTH, GH, insulin, IL-6 or IL-6/IL-6R gene will be injected into the defect site in a patient. The genes encoding each of the proteins may be placed in the same vector or in separate vectors.

### EXAMPLE 14

### Monitoring Effects of Gene Therapy in Patients

The repair site will be monitored radiographically every two weeks for a minimum of two years. It is anticipated that the defect site which receives the combination of OP-1+MPSF (delivered by either of the methods described in Examples 11, 12 and 13) will exhibit a faster rate of repair than that which receives OP-1 alone.

What is claimed is:

1. A method for inducing a progenitor cell to proliferate or differentiate comprising the step of contacting a progenitor cell with a nucleic acid encoding a morphogenic protein and a nucleic acid encoding a MPSF.

2. A method for inducing a progenitor cell to proliferate or differentiate comprising the steps of:
a) providing a vector comprising a nucleic acid encoding a morphogenic protein operably linked to an expression control sequence and a vector comprising a nucleic acid encoding a MPSF operably linked to an expression control sequence and
b) contacting said progenitor cell with said vectors.

3. The method according to claim 2, wherein the nucleic acid encoding the morphogenic protein and the nucleic acid encoding the MPSF are in the same vector.

4. The method according to claim 2, wherein the nucleic acid encoding the morphogenic protein and the nucleic acid encoding the MPSF are in separate vectors.

5. The method according to claim 1 or 2, wherein the progenitor cell is selected from the group consisting of a chondroblast, osteoblast, a tendon progenitor cell, a ligament progenitor cell and neuroblast.

6. The method according to claim 1 or 2, wherein the morphogenic protein is selected from the group consisting of OP-1 (BMP-7), OP-2, OP-3, COP-1, COP-3, COP-4, COP-5, COP-7, COP-16, BMP-2, BMP-3, BMP-3b, BMP-4, BMP-5, BMP-6, BMP-9, BMP-10, BMP-11, CDMP-3, BMP-12, CDMP-2, BMP-13, CDMP-1, BMP-14, BMP-15, BMP-16, BMP-17, BMP-18, GDF-1, GDF-2, GDF-3, GDF-5, GDF-6, GDF-7, GDF-8, GDF-9, GDF-10, GDF-11, GDF-12, MP121, dorsalin-1, DPP, Vg-1, Vgr-1, 60A protein, NODAL, UNIVIN, SCREW, ADMP, NEURAL, or fragments thereof.

7. The method according to claim 6, wherein the morphogenic protein is OP-1.

8. The method according to claim 1 or 2, wherein the MPSF is selected from the group consisting of insulin-like growth factor I (IGF-I), insulin-like growth factor II (IGF-II), fibroblast growth factor (FGF), growth hormone, insulin, and parathyroid hormone (PTH), IL-6 or IL-6/IL-6R.

9. The method of claim 8, wherein the MPSF is IGF-I.

10. The method of claim 8, wherein the MPSF is IL-6/IL-6R.

11. A method for inducing tissue formation, repairing a tissue defect or regenerating tissue, at a target locus in a mammal, comprising the step of administering to the target locus a nucleic acid encoding a morphogenic protein and a nucleic acid encoding a MPSF.

12. A method for inducing tissue formation, repairing a tissue defect or regenerating tissue, at a target locus in a mammal, comprising the steps of:
a) providing a vector comprising a nucleic acid encoding a morphogenic protein operably linked to an expression control sequence and a vector comprising a nucleic acid encoding a MPSF operably linked to an expression control sequence and
b) administering to the target locus said vector.

13. A method for inducing tissue formation, repairing a tissue defect or regenerating tissue, at a target locus in a mammal, comprising the steps of
a) providing a cultured host cell expressing a recombinant morphogenic protein and a recombinant MPSF, and
b) administering to the target locus the host cell expressing the recombinant morphogenic protein and the recombinant MPSF.

14. The method according to claims 11 or 12, wherein the nucleic acid encoding the morphogenic protein and the nucleic acid encoding the MPSF are in the same vector.

15. The method according to claims 11 or 12, wherein the nucleic acid encoding the morphogenic protein and the nucleic acid encoding the MPSF are in separate vectors.

16. The method according to claim 13, wherein the morphogenic protein and MPSF are expressed in separate cells.

17. The method according to claim 13, wherein the morphogenic protein and MPSF are expressed in the same cell.

18. The method according to any one of claims 11-13, wherein the target locus is selected from bone, cartilage, tendon, ligament and neural tissue.

19. The method according to any one of claims 11-13, wherein the morphogenic protein is selected from the group consisting of OP-1 (BMP-7), OP-2, OP-3, COP-1, COP-3, COP-4, COP-5, COP-7, COP-16, BMP-2, BMP-3, BMP-3b, BMP-4, BMP-5, BMP-6, BMP-9, BMP-10, BMP-11, CDMP-3, BMP-12, CDMP-2, BMP-13, CDMP-1, BMP-14, BMP-15, BMP-16, BMP-17, BMP-18, GDF-1, GDF-2, GDF-3, GDF-5, GDF-6, GDF-7, GDF-8, GDF-9, GDF-10, GDF-11, GDF-12, MP121, dorsalin-1, DPP, Vg-1, Vgr-1, 60A protein, NODAL, UNIVIN, SCREW, ADMP, NEURAL, or fragments thereof.

20. The method according to claim 19, wherein the morphogenic protein is OP-1.

21. The method according to any one of claims 11-13, wherein the MPSF is selected from the group consisting of insulin-like growth factor I (IGF-I), insulin-like growth factor II (IGF-II), fibroblast growth factor (FGF), growth hormone, insulin, parathyroid hormone (PTH), IL-6 or IL-6/IL-6R.

22. The method according to claim 21, wherein the MPSF is IGF-I.

23. The method according to claim 21, wherein the MPSF is IL-6/IL-6R.

24. A method of inducing tissue formation, repairing a tissue defect or regenerating tissue, by in vivo gene therapy, comprising the step of administering to target locus in a patient, a viral vector comprising a nucleotide sequence that encodes a morphogenic protein and a viral vector comprising a nucleotide sequence that encodes a MPSF so that the morphogenic protein and MPSF are expressed from the nucleotide sequence in the mammal in an amount sufficient to induce progenitor cells to proliferate or differentiate.

25. The method of claim 24, wherein the viral vector is selected from the group consisting of an adenoviral vector, a lentiviral vector, a baculoviral vector, an Epstein Barr viral vector, a papovaviral vector, a vaccinia viral vector, and a herpes simplex viral vector.

26. The method of claim 24, wherein the morphogenic protein is selected from the group consisting of OP-1 (BMP-7), OP-2, OP-3, COP-1, COP-3, COP-4, COP-5, COP-7, COP-16, BMP-2, BMP-3, BMP-3b, BMP-4, BMP-5, BMP-6, BMP-9, BMP-10, BMP-11, CDMP-3, BMP-12, CDMP-2, BMP-13, CDMP-1, BMP-14, BMP-15, BMP-16, BMP-17, BMP-18, GDF-1, GDF-2, GDF-3, GDF-5, GDF-6, GDF-7, GDF-8, GDF-9, GDF-10, GDF-11, GDF-12, MP121, dorsalin-1, DPP, Vg-1, Vgr-1, 60A protein, NODAL, UNIVIN, SCREW, ADMP, NEURAL, or fragments thereof.

27. The method according to claim 26, wherein the morphogenic protein is OP-1.

28. The method according to claim 24, wherein the MPSF is selected from the group consisting of insulin-like growth factor I (IGF-I), insulin-like growth factor II (IGF-II), fibroblast growth factor (FGF), growth hormone, insulin, parathyroid hormone (PTH), IL-6 or IL-6/IL-6R.

29. The method according to claim 28, wherein the MPSF is IGF-I.

30. The method accoridng to claim 28, wherein the MPSF is IL-6/IL-6R.


SEQUENCE LISTING (13 sequences)

SEQ ID NO: 1 - DNA, 1822 bp, Homo sapiens
  CDS (49)..(1341)
ggtgcgggcccggagcccggagcccgggtagcgcgtagagccggcgcgatgcacgtgcgctcactgcgagctgcggcgccgcacagcttcgtggcgctctgggcacccctgttcctgctgcgctccgccctggccgacttcagcctggacaacgaggtgcactcgagcttcatccaccggcgcctccgcagccaggagcggcgggagatgcagcgcgagatcctctccattttgggcttgccccaccgcccgcgcccgcacctccagggcaagcacaactcggcacccatgttcatgctggacctgtacaacgccatggcggtggaggagggcggcgggcccggcggccagggcttctcctacccctacaaggccgtcttcagtacccagggcccccctctggccagcctgcaagatagccatttcctcaccgacgccgacatggtcatgagcttcgtcaacctcgtggaacatgacaaggaattcttccacccacgctaccaccatcgagagttccggtttgatctttccaagatcccagaaggggaagctgtcacggcagccgaattccggatctacaaggactacatccgggaacgcttcgacaatgagacgttccggatcagcgtttatcaggtgctccaggagcacttgggcagggaatcggatctcttcctgctcgacagccgtaccctctgggcctcggaggagggctggctggtgtttgacatcacagccaccagcaaccactgggtggtcaatccgcggcacaacctgggcctgcagctctcggtggagacgctggatgggcagagcatcaaccccaagttggcgggcctgattgggcggcacgggccccagaacaagcagcccttcatggtggctttcttcaaggccacggaggtccacttccgcagcatccggtccacggggagcaaacagcgcagccagaaccgctccaagacgcccaagaaccaggaagccctgcggatggccaacgtggcagagaacagcagcagcgaccagaggcaggcctgtaagaagcacgagctgtatgtcagcttccgagacctgggctggcaggactggatcatcgcgcctgaaggctacgccgcctactactgtgagggggagtgtgccttccctctgaactcctacatgaacgccaccaaccacgccatcgtgcagacgctggtccacttcatcaacccggaaacggtgcccaagccctgctgtgcgcccacgcagctcaatgccatctccgtcctctacttcgatgacagctccaacgtcatcctgaagaaatacagaaacatggtggtccgggcctgtggctgccactagctcctccgagaattcagaccctttggggccaagtttttctggatcctccattgctcgccttggccaggaaccagcagaccaactgccttttgtgagaccttcccctccctatccccaactttaaaggtgtgagagtattaggaaacatgagcagcatatggcttttgatcagtttttcagtggcagcatccaatgaacaagatcctacaagctgtgcaggcaaaacctagcaggaaaaaaaaacaacgcataaagaaaaatggccgggccaggtcattggctgggaagtctcagccatgcacggactcgtttccagaggtaattatgagcgcctaccagccaggccacccagccgtgggaggaagggggcgtggcaaggggtgggcacattggtgtctgtgcgaaaggaaaattgacccggaagttcctgtaataaatgtcacaataaaacgaatgaatgaaaaaaaaaaaaaaaaaa

SEQ ID NO: 2 - protein, 431 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 3 - protein, 431 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETERISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 4 - protein, 96 aa, Artificial Sequence
  Synthetic amino acid sequence COP-5
LYVDFSDVGWDDWIVAPPGYQAFYCHGECPFPLADHFNSTNHAVVQTLVNSVNSKIPKACCVPTELSAISMLYLDENEKVVLKYNQEMVVEGCGCR

SEQ ID NO: 5 - protein, 96 aa, Artificial Sequence
  Synthetic amino acid sequence COP-7
LYVDFSDVGWNDWIVAPPGYHAFYCHGECPFPLADHLNSTNHAVVQTLVNSVNSKIPKACCVPTELSAISMLYLDENEKVVLKYNQEMVVEGCGCR

SEQ ID NO: 6 - protein, 102 aa, Artificial Sequence
  OP-X
  MOD_RES (1)..(102): Xaa is independently selected from a group of one or more specified amino acids as defined in the specification
  misc_feature (2)..(3): Xaa can be any naturally occurring amino acid
  misc_feature (9)..(9): Xaa can be any naturally occurring amino acid
  misc_feature (11)..(11): Xaa can be any naturally occurring amino acid
  misc_feature (16)..(16): Xaa can be any naturally occurring amino acid
  misc_feature (19)..(19): Xaa can be any naturally occurring amino acid
  misc_feature (23)..(23): Xaa can be any naturally occurring amino acid
  misc_feature (26)..(26): Xaa can be any naturally occurring amino acid
  misc_feature (35)..(35): Xaa can be any naturally occurring amino acid
  misc_feature (39)..(39): Xaa can be any naturally occurring amino acid
  misc_feature (41)..(41): Xaa can be any naturally occurring amino acid
  misc_feature (50)..(50): Xaa can be any naturally occurring amino acid
  misc_feature (52)..(52): Xaa can be any naturally occurring amino acid
  misc_feature (56)..(58): Xaa can be any naturally occurring amino acid
  misc_feature (60)..(61): Xaa can be any naturally occurring amino acid
  misc_feature (65)..(65): Xaa can be any naturally occurring amino acid
  misc_feature (71)..(71): Xaa can be any naturally occurring amino acid
  misc_feature (73)..(73): Xaa can be any naturally occurring amino acid
  misc_feature (75)..(75): Xaa can be any naturally occurring amino acid
  misc_feature (80)..(80): Xaa can be any naturally occurring amino acid
  misc_feature (82)..(82): Xaa can be any naturally occurring amino acid
  misc_feature (84)..(84): Xaa can be any naturally occurring amino acid
  misc_feature (87)..(87): Xaa can be any naturally occurring amino acid
  misc_feature (89)..(89): Xaa can be any naturally occurring amino acid
  misc_feature (91)..(91): Xaa can be any naturally occurring amino acid
  misc_feature (97)..(97): Xaa can be any naturally occurring amino acid
CXXHELYVXFXDLGWXDWXIAPXGYXAYYCEGECXFPLXSXMNATNHAIXQXLVHXXXPXXVPKXCCAPTXLXAXSVLYXDXSXNVXLXKXRNMVVXACGCH

SEQ ID NO: 7 - protein, 97 aa, Artificial Sequence
  Generic Sequence 7
  MOD_RES (1)..(97): Xaa is independently selected from a group of one or more specified amino acids as defined in the specification
  misc_feature (2)..(4): Xaa can be any naturally occurring amino acid
  misc_feature (6)..(8): Xaa can be any naturally occurring amino acid
  misc_feature (11)..(16): Xaa can be any naturally occurring amino acid
  misc_feature (18)..(21): Xaa can be any naturally occurring amino acid
  misc_feature (23)..(23): Xaa can be any naturally occurring amino acid
  misc_feature (26)..(26): Xaa can be any naturally occurring amino acid
  misc_feature (28)..(28): Xaa can be any naturally occurring amino acid
  misc_feature (30)..(31): Xaa can be any naturally occurring amino acid
  misc_feature (33)..(40): Xaa can be any naturally occurring amino acid
  misc_feature (44)..(60): Xaa can be any naturally occurring amino acid
  misc_feature (63)..(63): Xaa can be any naturally occurring amino acid
  misc_feature (65)..(72): Xaa can be any naturally occurring amino acid
  misc_feature (74)..(80): Xaa can be any naturally occurring amino acid
  misc_feature (82)..(82): Xaa can be any naturally occurring amino acid
  misc_feature (84)..(88): Xaa can be any naturally occurring amino acid
  misc_feature (90)..(90): Xaa can be any naturally occurring amino acid
  misc_feature (92)..(93): Xaa can be any naturally occurring amino acid
  misc_feature (95)..(95): Xaa can be any naturally occurring amino acid
  misc_feature (97)..(97): Xaa can be any naturally occurring amino acid
LXXXFXXXGWXXXXXXPXXXXAXYCXGXCXXPXXXXXXXXNHAXXXXXXXXXXXXXXXXXCCXPXXXXXXXXLXXXXXXXVXLXXXXXMXVXXCXCX

SEQ ID NO: 8 - protein, 102 aa, Artificial Sequence
  Generic Sequence 8
  MOD_RES (1)..(102): Xaa is independently selected from a group of one or more specified amino acids as defined in the specification
  misc_feature (2)..(5): Xaa can be any naturally occurring amino acid
  misc_feature (7)..(9): Xaa can be any naturally occurring amino acid
  misc_feature (11)..(13): Xaa can be any naturally occurring amino acid
  misc_feature (16)..(21): Xaa can be any naturally occurring amino acid
  misc_feature (23)..(26): Xaa can be any naturally occurring amino acid
  misc_feature (28)..(28): Xaa can be any naturally occurring amino acid
  misc_feature (31)..(31): Xaa can be any naturally occurring amino acid
  misc_feature (33)..(33): Xaa can be any naturally occurring amino acid
  misc_feature (35)..(36): Xaa can be any naturally occurring amino acid
  misc_feature (38)..(45): Xaa can be any naturally occurring amino acid
  misc_feature (49)..(65): Xaa can be any naturally occurring amino acid
  misc_feature (68)..(68): Xaa can be any naturally occurring amino acid
  misc_feature (70)..(77): Xaa can be any naturally occurring amino acid
  misc_feature (79)..(85): Xaa can be any naturally occurring amino acid
  misc_feature (87)..(87): Xaa can be any naturally occurring amino acid
  misc_feature (89)..(93): Xaa can be any naturally occurring amino acid
  misc_feature (95)..(95): Xaa can be any naturally occurring amino acid
  misc_feature (97)..(98): Xaa can be any naturally occurring amino acid
  misc_feature (100)..(100): Xaa can be any naturally occurring amino acid
  misc_feature (102)..(102): Xaa can be any naturally occurring amino acid
CXXXXLXXXFXXXGWXXXXXXPXXXXAXYCXGXCXXPXXXXXXXXNHAXXXXXXXXXXXXXXXXXCCXPXXXXXXXXLXXXXXXXVXLXXXXXMXVXXCXCX

SEQ ID NO: 9 - protein, 97 aa, Artificial Sequence
  Generic Sequence 9
  MOD_RES (1)..(97): Xaa is independently selected from a group of one or more specified amino acids as defined in the specification
  misc_feature (1)..(16): Xaa can be any naturally occurring amino acid
  misc_feature (18)..(24): Xaa can be any naturally occurring amino acid
  misc_feature (26)..(26): Xaa can be any naturally occurring amino acid
  misc_feature (28)..(28): Xaa can be any naturally occurring amino acid
  misc_feature (30)..(61): Xaa can be any naturally occurring amino acid
  misc_feature (63)..(63): Xaa can be any naturally occurring amino acid
  misc_feature (65)..(72): Xaa can be any naturally occurring amino acid
  misc_feature (74)..(93): Xaa can be any naturally occurring amino acid
  misc_feature (95)..(95): Xaa can be any naturally occurring amino acid
  misc_feature (97)..(97): Xaa can be any naturally occurring amino acid
XXXXXXXXXXXXXXXXPXXXXXXXCXGXCXXXXXXXXXXXXXXXXXXXXXXXXXXXXXXXXCXPXXXXXXXXLXXXXXXXXXXXXXXXXXXXXCXCX

SEQ ID NO: 10 - protein, 102 aa, Artificial Sequence
  Generic Sequence 10
  MOD_RES (1)..(102): Xaa is independently selected from a group of one or more specified amino acids as defined in the specification
  misc_feature (2)..(21): Xaa can be any naturally occurring amino acid
  misc_feature (23)..(29): Xaa can be any naturally occurring amino acid
  misc_feature (31)..(31): Xaa can be any naturally occurring amino acid
  misc_feature (33)..(33): Xaa can be any naturally occurring amino acid
  misc_feature (35)..(66): Xaa can be any naturally occurring amino acid
  misc_feature (68)..(68): Xaa can be any naturally occurring amino acid
  misc_feature (70)..(77): Xaa can be any naturally occurring amino acid
  misc_feature (79)..(98): Xaa can be any naturally occurring amino acid
  misc_feature (100)..(100): Xaa can be any naturally occurring amino acid
  misc_feature (102)..(102): Xaa can be any naturally occurring amino acid
CXXXXXXXXXXXXXXXXXXXXPXXXXXXXCXGXCXXXXXXXXXXXXXXXXXXXXXXXXXXXXXXXXCXPXXXXXXXXLXXXXXXXXXXXXXXXXXXXXCXCX

SEQ ID NO: 11 - protein, 5 aa, Artificial Sequence
  Consensus Sequence
  MOD_RES (1)..(5): Xaa is independently selected from a group of one or more specified amino acids as defined in the specification
  misc_feature (2)..(5): Xaa can be any naturally occurring amino acid
CXXXX

SEQ ID NO: 12 - DNA, 733 bp, Homo sapiens
  CDS (160)..(618)
cactgctaaattcagagcagattagagcctgcgcaatggaataaagtcctcaaaattgaaatgtgacattgctctcaacatctcccatctctctggatttccttttgcttcattattcctgctaaccaattcattttcagactttgtacttcagaagcaatgggaaaaatcagcagtcttccaacccaattatttaagtgctgcttttgtgatttcttgaaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaaggaagtacatttgaagaacgcaagtagagggagtgcaggaaacaagaactacaggatgtaggaagaccctcctgaggagtgaagagtgacatgccaccgcaggatcctttgctctgcacgagttacctgttaaactttggaacacctaccaaaaaataagtttgataacattt

SEQ ID NO: 13 - protein, 153 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM